(19) United States
(12) Patent Application Publication
(10) Pub. No.: US 2008/0031818 A1
(43) Pub. Date: Feb. 7, 2008

(54) Use of Modulators of a Novel Form of Muscle Selective Calcineurin Interacting Protein (Mcip-1-38) as a Treatment for Cardiovascular Diseases

(75) Inventors: Erik Bush, Erie, CO (US); David Hood, Thornton, CO (US)

Correspondence Address: FULBRIGHT & JAWORSKI L.L.P., 600 CONGRESS AVE., SUITE 2400, AUSTIN, TX, 78701, US

(73) Assignee: GILEAD COLORADO, INC.

(21) Appl. No.: 11/575,462

(22) PCT Filed: Sep. 19, 2005
(86) PCT No.: PCT/US05/33461

§ 371 Date: Jul. 3, 2007

Related U.S. Application Data
(60) Provisional application No. 60/611,150, filed on Sep. 17, 2004.

(51) Int. Cl. A61K 49/00; A61K 31/70; A61P 9/00; C12Q 1/02; G01N 33/53; C12Q 1/68; C07K 16/00; A61K 38/00

(52) U.S. Cl. 424/9.2; 435/29; 435/6; 435/7.21; 514/12; 514/2; 514/44; 514/789; 514/9; 530/387.9

(57) ABSTRACT

The present invention describes a novel form of the MCIP protein, a 38 kDa version (MCIP-1-38) that predominates in the human heart, the upregulation of which is strongly suggested for the treatment or prevention of heart disease. The present invention provides for methods of treating and preventing cardiovascular diseases, in particular pathological cardiac hypertrophy and chronic heart failure, by applying a modulator of MCIP-1-38. The present invention also provides for methods of screening to find modulators of MCIP-1-38 and inhibitors of cardiac hypertrophy and heart failure.

This application claims priority to U.S. Provisional Patent Application Ser. No. 60/611,150, filed Sep. 17, 2004, which is incorporated herein by reference in its entirety.

### BACKGROUND OF THE INVENTION

1. Field of the Invention

The present invention relates generally to the fields of developmental biology and molecular biology. More particularly, it concerns gene regulation and cellular physiology in cardiomyocytes. Specifically, the invention relates to the use of modulators of MCIP to enhance expression of a novel 38 kDa version of MCIP-1 described herein. It also relates to the use of said modulators to treat cardiac hypertrophy and heart failure, and to screening methods for finding modulators of 38 kDa MCIP-1 (MCIP-1-38).

2. Description of Related Art

Cardiovascular diseases encompass a wide variety of etiologies and have an equally wide variety of causative agents and interrelated players. Cardiac hypertrophy, for example, is an adaptive response of the heart to many forms of other cardiac disease, including hypertension, mechanical load abnormalities, myocardial infarction, valvular dysfunction, certain cardiac arrhythmias, endocrine disorders and genetic mutations in cardiac contractile protein genes. While the hypertrophic response is thought to be an initially compensatory mechanism that augments cardiac performance, sustained hypertrophy is maladaptive and frequently leads to ventricular dilation and the clinical syndrome of heart failure. Accordingly, cardiac hypertrophy has been established as an independent risk factor for cardiac morbidity and mortality.

One major downstream effector in a number of cardiovascular diseases is the calcium-dependent phosphatase calcineurin, which plays a critical role in the promotion of cardiac hypertrophy. Calcineurin, a serine/threonine protein phosphatase, plays a pivotal role in the developmental and homeostatic regulation of a wide variety of cell types (Klee et al., 1998; Crabteee, 1999). The interaction of calcineurin with transcription factors of the NFAT family following activation of the T cell receptor in leukocytes provides one of the earliest characterized examples of how calcineurin regulates gene expression (Rao et al., 1997). Changes in intracellular calcium promote binding of Ca²⁺/calmodulin to the catalytic subunit of calcineurin (CnA), thereby displacing an autoinhibitory region and allowing access of protein substrates to the catalytic domain. Activated calcineurin dephosphorylates the transcription factor NFAT, which then enters the nucleus and promotes hypertrophic gene expression. Dephosphorylation of NFAT by activated calcineurin is a necessary step for its translocation from the cytoplasm to the nucleus, allowing NFAT to bind DNA cooperatively with an AP1 heterodimer to activate transcription of genes encoding cytokines such as IL-2. This basic model of NFAT activation has been shown to transduce Ca2+ signals via calcineurin in many cell types and to control transcription of diverse sets of target genes unique to each cellular environment (Timmerman et al., 1996). In each case, NFAT acts cooperatively with other transcription factors that include proteins of the AP1 (Rao et al., 1997), cMAF (Ho et al., 1996), GATA (Mesaeli et al., 1999; Molkentin et al., 1998; Musaro et al., 1999), or MEF2 (Chin et al., 1998; Liu et al., 1997; Mao et al., 1999; Mao and Wiedmann, 1999) families. MEF-2 gene transcription has been shown to be both necessary and sufficient for the development of cardiac hypertrophy and heart failure via dilated cardiomyopathy (DCM) (Olson et al., 1995), implicating control of calcineurin as a very attractive therapeutic regimen for heart disease.

To that effect, studies of calcineurin signaling in striated myocytes of heart and skeletal muscle expanded the scope of important physiological and pathological events controlled by this ubiquitously expressed protein. Modulators of calcineurin were found to include immunophilins that were the targets of the immunosuppressant drugs cyclosporin A and FK-506, and two unrelated proteins, AKAP79 and cabin-1/cain. AKAP79 was shown to bind calcineurin in conjunction with protein kinase C and protein kinase A, serving as a scaffold for assembly of a large hetero-oligomeric signaling complex (Kashishian et al., 1998). Cabin-1/cain binds both calcineurin and the transcription factor MEF2 (Sun et al., 1998; Lai et al., 1998). As a consequence of cabin-1 overexpression, calcineurin activity was inhibited and MEF2 was sequestered in an inactive state. Inactivation of MEF-2, as previously mentioned, is anti-hypertrophic and protective for the heart (Olson et al., 1995).

These studies were limited by the fact that they were, as mentioned, not cardiospecific. Thus, it was an important finding when it was shown that forced expression of a constitutively active form of calcineurin in hearts of transgenic mice promotes cardiac hypertrophy and subsequent progression to dilated cardiomyopathy, heart failure, and death, in a manner that recapitulated features of human disease (Molkentin et al., 1998). Moreover, hypertrophy and heart failure in those animals, and in certain other animal models of cardiomyopathy, was prevented by administration of the calcineurin antagonist drugs cyclosporin A or FK-506 (Sussman et al., 1998). In skeletal muscles, calcineurin signaling was implicated both in hypertrophic growth stimulated by insulin-like growth factor-1 (Musaro et al., 1999; Semsarian et al., 1999), and in the control of specialized programs of gene expression that establish distinctive myofiber subtypes (Chin et al., 1998; Dunn et al., 1999). These observations stimulated interest in the therapeutic potential of modifying calcineurin activity selectively in muscle cells while avoiding unwanted consequences of altered calcineurin signaling in other cell types (Sigal et al., 1991).

The existence of calcineurin regulating compounds gave rise to potential therapeutic interventions targeting this molecule, which led to the discovery that MCIP was involved in the regulatory system surrounding calcineurin. MCIP is a protein that was previously characterized and described as a modulator of calcineurin (U.S. Patent Application 2002150953). It has also been shown that the activity of calcineurin in mammalian cardiac cells can be modulated by interactions with MCIP. MCIP was previously shown to exist as a 28-kDa protein with a variety of splice variants all from a single gene locus known as MCIP-1, but to date no known modulators of MCIP-1 have been shown to have therapeutic benefit (U.S. Patent Application 2002150953). Identifying new, more suitable candidates having the ability to modulate calcineurin function in cardiac tissue is an important goal of current research efforts, and led to the discovery presented herein that a novel form of MCIP, a 38 kDa form (MCIP-1-38), is not only the predominant form of MCIP in humans but that upregulation of MCIP-1-38 is cardiotonic and a potential treatment for cardiovascular diseases.

### SUMMARY OF THE INVENTION

Thus, in accordance with the present invention, there is provided a method of treating cardiovascular disease comprising first identifying a patient having cardiovascular disease and then administering to said patient a modulator of MCIP-1-38. The cardiovascular diseases may be selected from but not limited to one or more of pathologic cardiac hypertrophy, DCM, myocardial infarction (MI), primary or secondary pulmonary arterial hypertension (PPH, SPAH), chronic heart failure, atherosclerosis, and ischemic heart disease. The modulator used may be a small molecule, a peptide, a pharmaceutical, a protein, a cyclic peptide, or a nucleic acid. The nucleic acid may be an siRNA, an antisense RNA, or a viral expression vector.

In specific embodiments of the invention it is contemplated that a modulator of MCIP-1-38 will be administered to a subject. Said administration may comprise intravenous, oral, transdermal, sustained release, suppository, subcutaneous, sublingual, any form of direct injection or use of a stent, or by a gene therapy administration. The modulator may also be coupled with a second therapeutic regimen, which may be administered at the same time, before, or after the modulator. The second therapeutic may be selected from the group consisting of beta blockers, inotropes, phosphodiesterase inhibitors, diuretics, ACE-inhibitors, All antagonists, histone deacetylase inhibitors, Ca(++)-channel blockers, and endothelin receptor antagonists.

In certain embodiments, treating comprises the improvement of one or more symptoms of cardiac hypertrophy, where the one or more symptoms may be disease related hospitalizations, or a dysfunction in any one of exercise capacity, blood ejection volume, left ventricular end diastolic pressure, pulmonary capillary wedge pressure, cardiac output, decreased cardiac index, pulmonary artery pressures, left ventricular end systolic and diastolic dimensions, left and right ventricular wall stress, or wall tension, quality of life, disease-related morbidity and mortality.

In another embodiment of the invention, treating comprises improving one or more symptoms of heart failure. These one or more symptoms may be disease related hospitalizations, progressive remodeling of the heart, ventricular dilation, decreased cardiac output, impaired pump performance, arrhythmia, fibrosis, necrosis, energy starvation, or apoptosis.

In yet another embodiment of the invention there is provided a method of preventing cardiac hypertrophy or heart failure comprising identifying a patient at risk for cardiac hypertrophy or heart failure, and then administering to said patient a modulator of MCIP-1-38. It is contemplated that administering comprises intravenous oral, transdermal, sustained release, suppository, or sublingual administration of the modulator. In contemplated embodiments of the invention the patient at risk may exhibit one or more of long standing uncontrolled hypertension, atherosclerosis, uncorrected valvular disease, chronic angina and/or recent myocardial infarction. The modulator may consist of a small molecule, a peptide, a cyclic peptide, a protein, a pharmaceutical, or a nucleic acid.

In a further embodiment, there is provided a method of identifying a modulator of MCIP-1-38 comprising providing a cell, contacting said cell with a candidate substance, and measuring expression of MCIP-1-38 wherein an increase in expression of MCIP-1-38, as compared to expression in an untreated cell, identifies the candidate substance as a modulator of MCIP-1-38. In certain embodiments these cells are cardiomyocytes, and the MCIP-1-38 may be measured in intact cells by measuring the expression of an endogenous (native) gene or an exogenous gene expressed in the cells. In specific embodiments these cardiomyocytes may be neonatal rat ventricular myocytes (NRVM), adult rat cardiomyoctes, adult or neonatal mouse cardiomyocytes, or adult human cardiomyocytes. The cardiomyocytes may be located in an intact heart, and more specifically in a heart. The heart may be from a mammal, and more specifically from a rat, mouse or human. This method may be performed both as a small-scale screen and in high-throughput modes.

In yet another embodiment there is provided a method of identifying an inhibitor of heart failure or hypertrophy comprising first providing an MCIP-1-38 modulator, then treating a myocyte with said modulator, and finally measuring the expression of one or more cardiac hypertrophy or heart failure parameters, wherein a change in said one or more cardiac hypertrophy or heart failure parameters, as compared to one or more cardiac hypertrophy parameters in a myocyte not treated with said enhancer, identifies said modulator as an inhibitor of heart failure or cardiac hypertrophy. In specific embodiments the myocytes may be subjected to a stimulus that triggers a hypertrophic response in said one or more cardiac hypertrophy parameters. Said stimulus can be the expression of a transgene or treatment with a chemical agent.

In certain contemplated embodiments said one more cardiac hypertrophy parameters comprises the expression level of one or more target genes in said myocyte, wherein expression level of said one or more target genes is indicative of cardiac hypertrophy. Said one or more target genes may be selected from the group consisting of ANF, α-MyHC, β-MyHC, α-skeletal actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen.

In yet further embodiments the expression level is measured using a reporter protein coding region operably linked to a target gene promoter, and said reporter may be β-gal, or green fluorescent protein. The expression level may be measured using hybridization of a nucleic acid probe to a target mRNA or amplified nucleic acid product. In yet additional embodiments said one or more cardiac hypertrophy parameters comprises one or more aspects of cellular morphology, which may comprise sarcomere assembly, cell size, cellular fusion, or cell contractility. The myocytes may be isolated myocytes and they may comprise isolated but intact tissue. The myocytes can be cardiomyocytes and specifically NRVMs. The cardiomyocytes can be located in vivo in a functioning, intact heart muscle. Said functioning intact heart muscle may be subjected to a stimulus that triggers heart failure or a hypertrophic response in one or more cardiac hypertrophy parameters, and that stimulus can be aortic banding, rapid cardiac pacing, induced myocardial infarction, drug-containing osmotic minipumps, or transgene expression. In preferred embodiments, said one or more cardiac hypertrophy parameters comprises total protein synthesis, right ventricle ejection fraction, left ventricle ejection fraction, ventricular wall thickness, heart weight/body weight ratio, heart weight bone length ratios, or cardiac weight normalization measurement.

In yet another embodiment of the invention there is contemplated a polyclonal antibody to MCIP-1-38. In additional embodiments of the invention there are methods presented using this antibody to measure or analyze the levels of MCIP-1-38 in peripheral blood comprising (a) collecting PBMCs from an individual and (b) analyzing those PBMCs for MCIP-1-38. The analysis may be performed by enzyme-linked immunosorbant assay (ELISA) on cell lysate, or by an in situ version of the ELISA (cytoblot), or by standard Western blot analysis. In yet further embodiments there is provided a kit for these analyses that contains the antibody and appropriate reagents and controls to perform the analysis procedure.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Genomic organization of the human MCIP1 locus (adapted from Rothermel et al., 2003). The four known MCIP1 forms are encoded by transcripts that utilize unique first exons, but share common exons 5, 6 and 7. The molecular masses of the protein products of all four described MCIP1 forms are predicted to be approximately 23 kDa or less.

FIG. 2—(2A) The MCIP1.4 transcript encodes a protein product of approximately 28 kDa. Western analysis of mammalian COS cells transiently transfected with a vector expressing the human MCIP1.4 transcript. The MCIP1 antibody recognizes a single recombinant protein of approximately 28 kDa. (2B) The MCIP1 antibody recognizes two endogenous MCIP1 forms present in cardiomyocytes. Anti-MCIP1 Western analysis of protein isolated from neonatal rat ventricular myocytes (NRVM) reveals two endogenous MCIP1 forms: an approximately 28 kDa form that is inducible by exogenous calcineurin and suppressible by the calcineurin inhibitor cyclosporine A, and an approximately 38 kDa calcineurin-independent form (MCIP-1-38).

FIG. 3—Tissue distribution of endogenous MCIP1 forms. Anti-MCIP1 Western analysis of protein isolated from rat tissues confirm 28 kDa MCIP1 protein expression in striated muscle and brain, consistent with the known distribution of MCIP1.4 transcript. Endogenous MCIP-1-38 protein was broadly expressed, comprising the predominant MCIP1 form in muscle, brain, testis, lung and eye.

FIG. 4—Expression of endogenous MCIP1 protein forms in response to pressure overload hypertrophy in vivo. Representative anti-MCIP1 Western blot of total protein isolated from the left ventricles of rats subjected to sham operation (n=4) or transverse aortic banding (TAB, n=4). Expression of 28 kDa MCIP1 (MCIP1.4) increased under conditions of pressure overload; expression of MCIP-1-38 protein was unchanged.

FIG. 5—MCIP1 Western analysis of left ventricular protein samples isolated from six nonfailing and six failing (IDC) human hearts. (5A) MCIP-1-38 protein is the most abundantly expressed MCIP1 form in human heart (top panel); longer exposures reveal the lower abundance, calcineurin-dependent 28 kDa MCIP1 form (middle panel). Blot was reprobed with an antibody against calnexin/IP90 housekeeping protein as a loading control (lower panel). (5B) Semi-quantitative analysis of MCIP1 forms was done in the failing and non-failing human heart. Densitometric analysis of MCIP1 bands (normalized to IP90 loading controls) confirms a significant reduction in MCIP-1-38 expression in failing human hearts. The 28 kDa MCIP1 form showed a trend toward increased expression in failing hearts, but did not reach statistical significance. Data represent mean signal density ±S.E.

FIG. 6—Compounds that selectively increase expression of endogenous cardiac MCIP-1-38 protein. Compounds #1-3 are structurally related small molecules that increase MCIP-1-38 in cultured cardiomyocytes.

FIG. 7—Compounds #1, #2 and #3 selectively increases expression of MCIP-1-38 in cultured cardiomyocytes. (7A) Anti-MCIP1 Western of control NRVM and NRVM treated with adenovirus expressing activated calcineurin, phenylephrine (20 mM), or compound #1 (10 mM). Pro-hypertrophic stimuli like calcineurin or PE selectively induce expression of the calcineurin-regulated 28 kDa MCIP1 protein form. In contrast, compound #1 selectively induces expression of MCIP-1-38 protein form. (7B) Compound #2 also is shown to selectively increases expression of MCIP-1-38 in cultured cardiomyocytes in a concentration-dependent manner. Two independent experiments showing anti-MCIP1 Westerns of NRVM treated with increasing concentrations of compound #2 (top panels). Blots were reprobed with an antibody against calnexin/IP90 housekeeping protein as a loading control (lower panels). (7C) Compound #3 also is shown to selectively increases expression of MCIP-1-38 in cultured cardiomyocytes. Anti-MCIP1 Western of control NRVM and NRVM treated with phenylephrine (20 mM) or compound #3 (300 nM).

FIG. 8—Compound #3 exhibits little cellular toxicity in cultured cardiomyocytes and suppresses PE-dependent ANF release in cultured cardiomyocytes. (8A) NRVM were cultured for 48 hours in the presence of increasing concentrations of compound #3. Cellular toxicity was measured by quantitation of intracellular ATP. No cellular toxicity was observed at concentrations of 10 mM or less, with a small amount of toxicity observed at the 20 mM dose. Data points represent mean ATP signal expressed as a percentage of untreated controls ±S.E. (8B) NRVM were cultured for 48 hours in the presence or absence of the hypertrophic agonist PE. Increasing doses of compound #3 decreased PE-dependent expression of the hypertrophic marker ANF in a concentration-dependent manner. Unstimulated NRVM express low levels of ANF, and compound #3 reduced this basal ANF expression as well. Data points represent mean ANF signal expressed as a percentage of untreated (minus PE, minus compound) controls ±S.E.

FIG. 9—Compound #3 reduces PE-dependent hypertrophic increase in cardiomyocyte cell volume. NRVM were cultured for 48 hours in the presence or absence of the hypertrophic agonist PE. Exposure to PE increased cell volume by approximately 50%; 3 mM compound #3 significantly reduced cell volume in PE-treated myocytes. Data represent mean cell volumes expressed as a percentage of untreated controls ±S.E.

FIG. 10—Compound #3 alters myosin heavy chain expression and normalizes fetal gene expression. (10A) NRVM were cultured for 48 hours in the presence or absence of the hypertrophic agonist PE. Relative expression of endogenous beta myosin protein content was measured by cytoblot assay. Exposure to PE increased beta myosin protein expression by several fold; treatment with compound #3 significantly reduced beta myosin expression in PE-treated myocytes. Data points represent mean beta myosin signal (arbitrary light units) ±S.E. (10B) Compound #3 increases expression of the adult myosin isoform, alpha myosin heavy chain, in cultured cardiomyocytes. NRVM were cultured for 48 hours in the presence of increasing concentrations of compound #3. Relative expression of endogenous alpha myosin protein content was measured by cytoblot assay. Compound #3 increased cardiac alpha myosin expression in a concentration-dependent manner. 3 mM compound #3 increased cardiac alpha myosin expression by 50%. Data points represent mean alpha myosin signal expressed as a percent of untreated controls ±S.E. (10C) RNA dot blot analysis of control and PE-stimulated NRVM in the presence or absence of 3 mM compound #3 for a period of 48 hours (top panel). Bottom panel represents densitometric analysis of hybridization signals, expressed as a percent of GAPDH loading controls. Compound #3 normalized PE-dependent increases in expression of ANF and alpha skeletal actin mRNA.

### DESCRIPTION OF ILLUSTRATIVE EMBODIMENTS

### I. Cardiovascular Disease

Cardiovascular diseases are among the most common natural causes of death. The cardiovascular diseases include many serious diseases which involve the cardiac and vascular systems, such as atherosclerosis, ischemic heart diseases, cardiac failure, cardiac shock, arrhythmia, hypertension, cerebral vascular diseases and peripheral vascular diseases.

Atherosclerosis most often occurs as a complication of hyperlipidemia and can be treated with antihyperlipidemic agents. Ischemic heart disease, cardiac failure, cardiac shock, cerebral vascular disease, peripheral vascular disease, hypertension, arrhythmia and arteriosclerosis may be fatal because ischemia develops in various organs such as the heart, brain and the walls of blood vessels. The ischemia damages the organs in which it develops because it impairs the functions of mitochondria that produce adenosine triphosphate (ATP), which is a phosphate compound with high energy potential serving as an energy source for the constituent cells of these organs. The resulting functional damage of organs can be fatal if it occurs in vital organs such as the heart, brain and blood vessels. It is therefore important for treating these diseases to restore the functional impairment of mitochondria caused by ischemia. Antiarrhythmic agents have been used to treat ischemic heart disease and arrhythmia, but their use with patients with possible cardiac failure has been strictly limited because these agents may cause cardiac arrest by their cardiodepressant effects.

The cardiovascular diseases named above may develop independently, but more often than not they occur in various combinations. For example, ischemic heart diseases are frequently accompanied by arrhythmia and cardiac failure, and complications of cerebrovascular disorder with hypertension are well known. Atherosclerosis is often complicated by one or more cardiovascular diseases and can make the patient seriously ill.

Cardiovascular diseases, which are often complicated by other cardiovascular diseases, have often been treated with a combination of multiple drugs, each of which is specific for a single disease. However, drug-therapy employing multiple agents presents problems for both doctors and patients: doctors always consider compatibilities and contraindications of drugs, and patients suffer both mental and physical distresses due to complicated administration of various drugs and high incidence of adverse reactions. Therefore, it has long been desired to develop a therapeutic agent that has overall pharmacological activities against cardiovascular diseases and which can be employed in the treatment of these diseases with high efficacy.

A. Hypertrophy, DCM, Chronic Heart Failure

As discussed above, cardiovascular diseases encompass a huge array of syndromes and disorders, all of which combined are among the leading causes of death worldwide. Heart failure by itself is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce heart failure, the symptoms of cardiomyopathy are those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

The currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

In light of the limitations of the current therapies, the inventors describe herein the identification of a novel form of MCIP, MCIP-1-38, which is found in both healthy and diseased human hearts and the modulation of which is cardiotonic. Thus, and in accordance with the present invention, the inventors describe herein a novel therapeutic method for treating cardiovascular disease that constitutes enhancing or upregulating MCIP-1-38 expression.

B. Primary and Secondary Pulmonary Arterial Hypertension

1. Primary Pulmonary Hypertension (PPH) PPH is a rare disease characterized by elevated pulmonary artery pressure with no apparent cause. PPH is also termed precapillary pulmonary hypertension or idiopathic pulmonary arterial hypertension. The diagnosis is usually made after excluding other known causes of pulmonary hypertension (Dresdale et al., 1951).

The pathophysiology of PPH is poorly understood. It is believed that an insult of some kind (e.g., hormonal, mechanical, other) to the endothelium first occurs, resulting in a cascade of events characterized by vascular scarring, endothelial dysfunction, and intimal and medial (smooth muscle) proliferation. At least 10-15% of patients with PPH have a familial form, which has only recently been characterized. Some cases may be related to sporadic genetic defects (Oudiz et al., 2004).

Early in the disease, as the pulmonary artery pressure increases and the right ventricle must perform extra work, thrombotic pulmonary arteriopathy occurs. Thrombotic pulmonary arteriopathy is characterized by in situ thrombosis of small muscular arteries of the pulmonary vasculature. In later stages, as the pulmonary pressure continues to rise, plexogenic pulmonary arteriopathy develops. This is characterized by a remodeling of the pulmonary vasculature with intimal fibrosis and replacement of normal endothelial structure (Oudiz et al., 2004).

PPH has no cure, and left untreated, PPH leads inexorably leads to right-sided heart failure and death. The overall survival rate in one study was approximately 30% at 3 years. Prior to the 1990s, therapeutic options were limited. The recent emergence of prostacyclin analogues, endothelin receptor antagonists, and other novel drug therapies has greatly improved the outlook for patients with PPH and PPH-like diseases, but no one treatment is currently considered state of the art. As with the aforementioned cardiovascular diseases, modulation of and upregulation of MCIP-1-38 could be an attractive therapeutic alternative to the current modalities.

2. Secondary or PAH

Secondary pulmonary artery hypertension (SPAH) is defined as a pulmonary artery systolic pressure higher than 30 mm Hg or a pulmonary artery mean pressure higher than 20 mm Hg secondary to either a pulmonary or a cardiac disorder. If no etiology can be identified, the pulmonary arterial hypertension (PAH) is termed primary pulmonary hypertension. An increased volume of pulmonary blood flow, escalating resistance in the pulmonary vascular bed, or an elevation in pulmonary venous pressure can induce the rise in pulmonary arterial pressure (Oudiz et al., 2004).

Cardiac disorders, pulmonary disorders, or both in combination are the most common causes of secondary pulmonary hypertension. Cardiac diseases produce pulmonary hypertension via volume or pressure overload, although subsequent intimal proliferation of pulmonary resistance vessels adds an obstructive element. Perivascular parenchymal changes along with pulmonary vasoconstriction are the mechanism of pulmonary hypertension in respiratory diseases.

Therapy for secondary pulmonary hypertension is targeted at the underlying cause and its effects on the cardiovascular system. Novel therapeutic agents undergoing clinical trials have led to the possibility of specific therapies for these once untreatable disorders. There are three predominant pathophysiologic mechanisms which may be involved in the pathogenesis of SPAH, (1) hypoxic vasoconstriction, (2) decreased area of the pulmonary vascular bed, and (3) volume/pressure overload (Oudiz et al., 2004).

Chronic hypoxemia causes pulmonary vasoconstriction by a variety of actions on pulmonary artery endothelium and smooth muscle cells, including down-regulation of endothelial nitric oxide synthetase and reduced production of the voltage-gated potassium channel alpha subunit. Chronic hypoxemia leading to pulmonary hypertension can occur in patients with chronic obstructive pulmonary disease (COPD), high-altitude disorders, and hypoventilation disorders (e.g., obstructive sleep apnea).

COPD is the most common cause of SPAH. These patients have worse 5-year survival rates, more severe ventilation perfusion mismatch, and nocturnal or exercise-induced hypoxemia. Other disorders, such as obstructive sleep apnea, neuromuscular disorders, and disorders of the chest wall, may lead to hypoxic pulmonary vasoconstriction and eventually SPAH (Oudiz et al., 2004).

A variety of causes may decrease the cross-sectional area of the pulmonary vascular bed, primarily due to disease of the lung parenchyma. The pulmonary arterial pressure rises only when the loss of the pulmonary vessels exceeds 60% of the total pulmonary vasculature. Patients with collagen vascular diseases have a high incidence of SPAH, particularly patients with systemic scleroderma or CREST (calcinosis cutis, Raynaud phenomenon, esophageal motility disorder, sclerodactyly, and telangiectasia) syndrome. A mild-to-moderate elevation in mean pulmonary artery pressure occurs secondary to acute pulmonary embolism. The peak systolic pressures usually do not rise above 50 mm Hg, and they generally normalize following appropriate therapy. Chronic pulmonary emboli can result in progressive PAH. HIV infection and several drugs and toxins are also known to cause PAH (Oudiz et al., 2004).

Disorders of the left heart may cause SPAH, resulting from volume and pressure overload. Pulmonary blood volume overload is caused by left-to-right intracardiac shunts, such as in patients with atrial or ventricular septal defects. Left atrial hypertension causes a passive rise in pulmonary arterial systolic pressure in order to maintain a driving force across the vasculature. Over time, persistent pulmonary hypertension accompanied by vasculopathy occurs. This may occur secondary to left ventricular dysfunction, mitral valvular disease, constrictive pericarditis, aortic stenosis, and cardiomyopathy (Oudiz et al, 2004).

Pulmonary venous obstruction is a rare cause of pulmonary hypertension. This may occur secondary to mediastinal fibrosis, anomalous pulmonary venous drainage, or pulmonary venoocclusive disease.

Increasing pulmonary arterial pressure is associated with a progressive decline in survival for patients with COPD or other interstitial lung diseases. The prognosis of patients with SPAH is variable and depends on the severity of hemodynamic derangement and the underlying primary disorder. Patients with severe pulmonary hypertension or right heart failure survive approximately 1 year. Patients with moderate elevations in pulmonary artery pressure (mean pressure<55 mm Hg) and preserved right heart function have a median survival of 3 years from diagnosis.

Although treatment of secondary pulmonary hypertension consists primarily of that necessary for the underlying disease, several medications and oxygen are used in different clinical settings. Currently, definite proof of effectiveness is lacking for several of these treatments (Oudiz et al., 2004). As such, there is a need for better medications for the treatment of PAH and modulators of MCIP-1-38 present just such an opportunity.

C. Myocardial Infarction & Ischemic Heart Disease

Ischemic heart disease is the leading cause of death in industrialised countries. The management of ischemic heart disease essentially relies upon one of three strategies, comprising medical therapy, percutaneous transluminal procedures, such as coronary angioplasty and atherectomy, and coronary artery bypass grafting. Although medical treatment remains the mainstay of anti-ischemic therapy, many patients undergo additional, invasive therapy in an attempt to restore coronary blood flow. However, there is increasingly intense discussion regarding not only the relative merits of these therapeutic approaches but also the point within the management of ischaemic heart disease at which they should be applied and the type of patient for which each is more appropriate.

Acute myocardial infarction (MI) strikes the majority of sufferers without prior warning and in the absence of clinically detectable predisposing risk factors (for a full review, see Braunwald, 1997). When patients come to the intensive unit in a hospital showing symptoms of acute MI, the diagnosis for acute MI requires that the patients must have (1) an increase in the plasma concentration of cardiac enzymes and (2) either a typical clinical presentation and/or typical ECG changes. Either of the following parameters will fulfill the requirement for an increase in cardiac enzymes: (1) Total creatine-kinase (CI) at least 2 times the upper limit of the normal range, or (2) CK-MB (muscle-brain) above the upper limit of the normal range and at least 5% of the normal CK. If total CK or CK-MB is not available, the following will be accepted in the fulfillment of the criteria for acute MI: (1) Troponin T at least 3 times the upper limit of the normal range; (2) Troponin I at least 3 times the upper limit of the normal range. The use of Troponin T as a serum marker for MI is disclosed in Murthy and Karmen (1997). The analytical performance and clinical utility of a sensitive immunoassay for determination of cardiac Troponin I can be taken from Davies et al. (1997).

Typical ECG changes include evolving ST-segment or T-wave changes in two or more contiguous ECG leads, the development of new pathological Q/QS waves in two or more contiguous ECG leads, or the development of new left bundle branch block.

Secondary prevention, namely the implementation of therapy to postpone further coronary events, thus continues to remain the major goal of prophylactic drug therapy in these patients. Survivors of acute MI are at moderate risk of recurrent infarction or cardiac death. Morbidity and mortality following an MI may be related to arrhythmias, to left ventricular dysfunction, and to recurrent MI. Because aspirin had a significant protective effect in secondary prevention of vascular disease, the possible benefit of aspirin in primary prevention was tested. However, several studies have shown that only a limited percent of the population at risk really benefits from aspirin therapy (Cairns et al., 1995). Thus, while the concept of secondary prevention of reinfarction and death after recovery from an MI has been actively investigated for several decades, there have been problems in proving the efficacy of various interventions. These problems have been related both to the ineffectiveness of certain strategies and to the difficulty in proving a benefit as mortality and morbidity have improved following MI.

The development of the AT (1) receptor antagonists provided, in addition to the ACE inhibitors, a new, more specific pharmacological tool to inhibit the renin-angiotensin cascade. However, there are distinguishing features between AT (1) receptor antagonists and ACE inhibitors that highlight their current limitations. One is manifested by the concomitant potentiation of bradykinin produced by ACE inhibitors, since the kinase II and converting enzyme are one in the same. The bradykinin related mechanism mediated through nitric oxide, prostaglandins, and endothelially derived hyper-polaring factor may be responsible for a different clinical effect of ACE inhibitors. Furthermore, the effect of the AT (2) is not yet clear, as an inhibition of the AT (1) receptor leads to an increase of AT (2).

Thus, the current treatments available to treat and prevent MI are severely limited. Improving the function of the heart and reversing early remodeling could be protective against subsequent m.i., and any agent that could prevent the development of cardiac hypertrophy would certainly be beneficial post-m.i. As such, and in accordance with the present invention, the inventors disclose the use of modulators of MCIP-1-38 to both treat patients post-MI as well as be used prophylactically in patients who are at risk of developing MI.

### II. MCIP and its Role in Cardiovascular Disease

A. Calcineurin

Calcineurin is a ubiquitously expressed serine/threonine phosphatase that exists as a heterodimer, comprised of a 59 kD calmodulin-binding catalytic A subunit and a 19 kD Ca(++)-binding regulatory B subunit (Stemmer and Klee, 1994; Su et al., 1995). Calcineurin is uniquely suited to mediate the prolonged hypertrophic response of a cardiomyocyte to Ca(++) signaling because the enzyme is activated by a sustained Ca(++) plateau and is insensitive to transient Ca(++) fluxes as occur in response to cardiomyocyte contraction (Dolmetsch et al., 1997).

Activation of calcineurin is mediated by binding of Ca(++) and calmodulin to the regulatory and catalytic subunits, respectively. Previous studies showed that over-expression of calmodulin in the heart also results in hypertrophy, but the mechanism involved was not determined (Gruver et al., 1993). It is now clear that calmodulin acts through the calcineurin pathway to induce the hypertrophic response. Calcineurin has been shown previously to dephosphorylate NF-AT3, which subsequently acts on the transcription factor MEF-2 (Olson et al., 1995). Once this event occurs, MEF-2 activates a variety of genes known as fetal genes, the activation of which inevitably results in hypertrophy (see below).

CsA and FK-506 bind the immunophilins cyclophilin and FK-506-binding protein (FKBP12), respectively, forming complexes that bind the calcineurin catalytic subunit and inhibit its activity. CsA and FK-506 block the ability of cultured cardiomyocytes to undergo hypertrophy in response to AngII and PE. Both of these hypertrophic agonists have been shown to act by elevating intracellular Ca(++), which results in activation of the PKC and MAP kinase signaling pathways (Sadoshima et al., 1993; Sadoshima and Izumo, 1993; Kudoh et al., 1997; Yamazaki et al., 1997, Zou et al., 1996). CsA does not interfere with early signaling events at the cell membrane, such as PI turnover, Ca(++) mobilization, or PKC activation (Emmel et al., 1989). Thus, its ability to abrogate the hypertrophic responses of AngII and PE suggests that calcineurin activation is an essential step in the AngII and PE signal transduction pathways, and its action has been shown to be mediated through transcription factor NF-AT3.

B. NF-AT3

NF-AT3 is a member of a multigene family containing four members, NF-ATc, NF-ATp, NF-AT3, and NF-AT4 (McCaffery et al., 1993; Northrup et al., 1994; Hoey et al., 1995; Masuda et al., 1995; Park et al., 1996; Ho et al., 1995). These factors bind the consensus DNA sequence GGAAAAT as monomers or dimers through a Rel homology domain (RHD) (Rooney et al., 1994; Hoey et al., 1995). Three of the NF-AT genes are restricted in their expression to T-cells and skeletal muscle, whereas NF-AT3 is expressed in a variety of tissues including the heart (Hoey et al., 1995). For additional disclosure regarding NF-AT proteins the skilled artisan is referred to U.S. Pat. No. 5,708,158, specifically incorporated herein by reference.

NF-AT3 is a 902-amino acid protein with a regulatory domain at its amino-terminus that mediates nuclear translocation and a Rel-homology domain near its carboxyl-terminus that mediates DNA binding. There are three different steps involved in the activation of NF-AT proteins, namely, dephosphorylation, nuclear localization and an increase in affinity for DNA. In resting cells, NFAT proteins are phosphorylated and reside in the cytoplasm. These cytoplasmic NF-AT proteins show little or no DNA affinity. Stimuli that elicit calcium mobilization result in the rapid dephosphorylation of the NF-AT proteins and their translocation to the nucleus. The dephosphorylated NF-AT proteins show an increased affinity for DNA. Each step of the activation pathway may be blocked by CsA or FK506. This implies, and earlier studies have shown, that calcineurin is the protein responsible for NF-AT activation (Olson et al., 1995).

Thus, many of the changes in gene expression in response to calcineurin activation are mediated by members of the NF-AT family of transcription factors, which translocate to the nucleus following dephosphorylation by calcineurin. Many observations support the conclusion that NF-AT also is an important mediator of cardiac hypertrophy in response to calcineurin activation. NF-AT activity is induced by treatment of cardiomyocytes with AngII and PE. This induction is blocked by CsA and FK-506, indicating that it is calcineurin-dependent. NF-AT3 synergizes with GATA4 to activate the cardiac specific BNP promoter in cardiomyocytes. Also, expression of activated NF-AT3 in the heart is sufficient to bypass all upstream elements in the hypertrophic signaling pathway and evoke a hypertrophic response.

Prior work demonstrates that the C-terminal portion of the Rel-homology domain of NF-AT3 interacts with the second zinc finger of GATA4, as well as with GATA5 and GATA6, which are also expressed in the heart. The crystal structure of the DNA binding region of NF-ATc has revealed that the C-terminal portion of the Rel-homology domain projects away from the DNA binding site and also mediates interaction with AP-1 in immune cells (Wolfe et al., 1997).

According to one model previously proposed, hypertrophic stimuli such as AngII and PE, which lead to an elevation of intracellular Ca(++), result in activation of calcineurin. NF-AT3 within the cytoplasm is dephosphorylated by calcineulin, enabling it to translocate to the nucleus where it can interact with GATA4, and then activate the transcription factor MEF-2, a family of transcription factors that are normally repressed by a tight association with class II HDAC's.

Results of previous work have shown that calcineurin activation of NF-AT3 regulates hypertrophy in response to a variety of pathologic stimuli and suggests a sensing mechanism for altered sarcomeric function. Of note, there are several familial hypertrophic cardiomyopathies (FHC) caused by mutations in contractile protein genes, which result in subtle disorganization in the fine crystalline-like structure of the sarcomere (Watkins et al., 1995; Vikstrom and Leinwand, 1996). It is unknown how sarcomeric disorganization is sensed by the cardiomyocyte, but it is apparent that this leads to altered Ca(++) handling (Palmiter and Solaro, 1997; Botinelli et al., 1997; Lin et al., 1996). Calcineurin, as discussed above, is one of the sensing molecules that couples altered Ca(++) handling associated with FHC with cardiac hypertrophy and heart failure. As has been mentioned previously, these studies and the relation between NF-AT3 and calcineurin led to a search for molecules or agents that could modulate calcineurin's activation of NF-AT3 specifically in cardiac cells. MCIP-1 was discovered to be such a calcineurin modulating protein.

C. MCIP-1 and MCIP-1-38

The importance of MCIP was unraveled, as mentioned previously, during efforts to discover modulators of calcineurin in relation to calcineurin's role in heart failure and cardiovascular diseases such as hypertrophy. One class of endogenous calcineurin inhibitors are the modulatory calcineurin-interacting proteins MCIP1, 2 and 3 (previously known as DSCR1, ZAKI-4 and DSCR1L), a recently described family of inhibitory proteins expressed primarily in striated muscle and nervous tissue (reviewed in Rothermel et al., 2003). MCIP-1 is unique among endogenous calcineurin inhibitors in that activated calcineurin strongly induces expression of a splice variant of MCIP-1 mRNA in transgenic mouse hearts and cultured myocytes, suggesting that MCIP-1 protein functions as a feedback inhibitor, protecting the cardiac myocyte from unchecked calcineurin activity.

Enhanced MCIP-1 expression may be a common response of the heart to a variety of hypertrophic stimuli, since pressure overload, mechanical strain, and hypertrophic agonists have all been demonstrated to increase cardiac expression of MCIP-1 mRNA. Furthermore, overexpression of MCIP-1 in the hearts of transgenic mice attenuated the hypertrophic response induced by activated calcineurin, β-adrenergic stimulation, exercise training, pressure overload and myocardial infarction, supporting a role for calcineurin-dependent signaling in diverse forms of cardiac hypertrophy.

MCIP-1 directly binds and inhibits calcineurin, functioning as an endogenous feedback inhibitor of calcineurin activity. Overexpression of MCIP-1 in the hearts of transgenic animals is anti-hypertrophic; MCIP-1 attenuates in vivo models of both calcineurin-dependent hypertrophy (Rothermel et al., 2001) and pressure-overload-induced hypertophy (Hill et al., 2002). MCIP-1 also acts as a substrate for phosphoryalation by MAPK and GSK-3, and calcineurin's phosphatase activity. Residues 81-177 of MCIP-1 retain the calcineurin inhibitory action.

Binding of MCIP-1 to calcineurin does not require calmodulin, nor does MCIP-1 interfere with calmodulin binding to calcineurin. This suggests that the surface of calcineurin to which MCIP-1 bindings does not include the calmodulin binding domain. In contrast, the interaction of MCIP-1 with calcineurin is disrupted by FK506:FKBP or cyclosporin:cyclophylin, indicating that the surface of calcineurin to which MCIP-1 binds overlaps with that required for the activity of immunosuppressive drugs.

The use of alternative promoters at the MCIP-1 locus gives rise to at least four different transcripts; the four previously identified and known MCIP-1 transcripts (MCIP1.1, 1.2, 1.3 and 1.4) are distinguished by a unique first exon, followed by three common exons (reviewed in Rothermel et al., 2003). The MCIP-1.4 transcript is the best-studied splice variant of this locus. Fifteen NFAT binding sites within the MCIP-1.4 promoter facilitate enhanced expression of MCIP-1.4 in response to calcineurin activation. All current transgenic MCIP-1 mouse studies utilize mice that overexpress the protein encoded by the MCIP-1.4 transcript.

The open reading frames of MCIP-1.1, 1.2, 1.3 and 1.4 transcripts have been predicted to encode proteins of molecular masses 22.7 kDa, 21.4 kDa, 19.9 kDa and 22.8 kDa, respectively. Until recently, however, examination of endogenous MCIP-1 protein has not been possible due to the lack of specific antibodies. The inventors have developed one of the first specific antibodies for MCIP-1 proteins, and have confirmed by peptide mass fingerprinting that the calcineurin-regulated MCIP-1.4 transcript gives rise to an endogenous protein of approximately 28 kDa. The MCIP-1 antibody also recognizes an endogenous, previously undescribed or predicted calcineurin-independent version of the protein of approximately 38 kDa (MCIP-1-38), significantly larger than the predicted molecular mass of any previously described transcript. This observation was subsequently confirmed by an independent group (see Genesca et al., Biochem. J. 2003, 374, 567-575); the authors of the study speculate that this larger band represents protein encoded by the MCIP-1.1 transcript. The inventors have purified endogenous human MCIP-1-38 protein and have confirmed by peptide mass fingerprinting that it is a product of the MCIP-1 locus. Diagnostic peptides from all three common MCIP-1 exons were recovered from the 38 kDa band (see Examples), however, no peptides recovered from the MCIP-1-38 corresponded to the unique first exon of the MCIP-1.1 transcript. This observation, along with the disparity in the predicted vs actual molecular masses, lead the inventors to conclude that MCIP-1-38 protein is encoded by a novel MCIP-1 transcript.

The inventors herein show that the MCIP-1-38 has a different tissue distribution than the calcineurin-regulated 28 kDa MCIP-1.4 protein, and that the 38 kDa protein is the most abundant form of MCIP-1 in the human left ventricle. We further demonstrate that unlike 28 kDa MCIP-1.4 protein, endogenous MCIP-1-38 protein is not induced in rodent models of cardiac hypertrophy, suggesting that the larger isoform is regulated independently of calcineurin. Finally, three compounds have been identified in a screen for small molecules that are capable of increasing MCIP-1-38 expression selectively, and are, in accordance with the current invention, capable of suppressing cardiac hypertrophy.

D. MEF2

As mentioned above, NF-AT3 activation by Calcineurin leads to the activation of another family of transcription factors, the monocyte enhancer factor-2 family (MEF2), which are known to play an important role in morphogenesis and myogenesis of skeletal, cardiac, and smooth muscle cells (Olson et al., 1995). Thus, inhibition of calcineurin through MCIP-1-38 would likely alter or abrogate the activation of MEF2, explaining at least in part the anti-hypertrophic properties of MCIP-1-38.

MEF2 factors are expressed in all developing muscle cell types, binding a conserved DNA sequence in the control regions of the majority of muscle-specific genes. Of the four mammalian MEF2 genes, three (MEF2A, MEF2B and MEF2C) can be alternatively spliced, which have significant functional differences (Brand, 1997; Olson et al., 1995). These transcription factors share homology in an N-terminal MADS-box and an adjacent motif known as the MEF2 domain. Together, these regions of MEF2 mediate DNA binding, homo- and heterodimerization, and interaction with various cofactors, such as the myogenic bHLH proteins in skeletal muscle. Additionally, biochemical and genetic studies in vertebrate and invertebrate organisms have demonstrated that MEF2 factors regulate myogenesis through combinatorial interactions with other transcription factors.

Loss-of-function studies indicate that MEF2 factors are essential for activation of muscle gene expression during embryogenesis. The expression and functions of MEF2 proteins are subject to multiple forms of positive and negative regulation, serving to fine-tune the diverse transcriptional circuits in which the MEF2 factors participate. MEF-2 is bound in an inactive form in the healthy heart by class II HDACS (see supra), and when MEF-2 is activated it is released from the HDAC and activates the fetal gene program that is so deleterious for the heart.

D. Histone Deacetylase

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

More than seventeen different HDACs have been cloned from vertebrate organisms. The first three human HDACs identified were HDAC 1, HDAC 2 and HDAC 3 (termed class I human HDACs), and HDAC 8 (Van den Wyngaert et al., 2000). Class II human HDACs, HDAC 4, HDAC 5, HDAC 6, HDAC 7, HDAC 9, and HDAC 10 (Kao et al., 2000) have been cloned and identified (Grozinger et al., 1999; Zhou et al. 2001; Tong et al., 2002). Additionally, HDAC 11 has been identified but not yet classified as either class I or class II (Gao et al., 2002) and there is a new class of HDACs known as class III. HDACs 4, 5, 7, 9 and 10 have a unique amino-terminal extension not found in other HDACs. This amino-terminal region contains the MEF2-binding domain. HDACs 4, 5 and 7 have been shown to be involved in the regulation of cardiac gene expression and in particular embodiments, repressing MEF2 transcriptional activity. The exact mechanism in which class II HDAC's repress MEF2 activity is not completely understood. One possibility is that HDAC binding to MEF2 inhibits MEF2 transcriptional activity, either competitively or by destabilizing the native, transcriptionally active MEF2 conformation. It also is possible that class II HDAC's require dimerization with MEF2 to localize or position HDAC in a proximity to histones for deacetylation to proceed. No matter how HDACs inhibit MEF-2, calcium signaling mediated through calcineurin is responsible for freeing HDACs from MEF-2, leading to activation of the fetal gene program. As such, while it may be useful to inhibit HDACs themselves, MCIP-1-38 could be mediating its antihypertrophic effect by modulating calcineurin dependent activation of HDACs.

A variety of inhibitors for histone deacetylase have been identified. The proposed uses range widely, but primarily focus on cancer therapy. See Saunders et al (1999); Jung et al. (1997); Jung et al. (1999); Vigushin et al. (1999); Kim et al. (1999); Kitazomo et al. (2001); Vigushin et al. (2001); Hoffmann et al. (2001); Kramer et al. (2001); Massa et al. (2001); Komatsu et al. (2001); Han et al. (2000). Such therapy is the subject of NIH sponsored clinical trials for solid and hematological tumors. HDAC's also increase transcription of transgenes, thus constituting a possible adjunct to gene therapy. (Yamano et al., 2000; Su et al., 2000).

HDACs can be inhibited through a variety of different mechanisms—proteins, peptides, and nucleic acids (including antisense, RNAi molecules, and ribozymes). Methods are widely known to those of skill in the art for the cloning, transfer and expression of genetic constructs, which include viral and non-viral vectors, and liposomes. Viral vectors include adenovirus, adeno-associated virus, retrovirus, vaccina virus and herpesvirus.

Perhaps the most widely known small molecule inhibitor of HDAC function is Trichostatin A, a hydroxamic acid. It has been shown to induce hyperacetylation and cause reversion of ras transformed cells to normal morphology (Taunton et al., 1996) and induces immunosuppression in a mouse model (Takahashi et al., 1996). It is commercially available from a variety of sources including BIOMOL Research Labs, Inc., Plymouth Meeting, Pa.

The following references, incorporated herein by reference, all describe HDAC inhibitors that may find use in the present invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. 2002/256221; U.S. 2002/103192; U.S. 2002/65282; U.S. 2002/61860; WO 02/51842; WO 02/50285; WO 02/46144; WO 02/46129; WO 02/30879; WO 02/26703; WO 02/26696; WO 01/70675; WO 01/42437;WO 01/38322; WO 01/18045; WO 01/14581; Furumai et al. (2002); Hinnebusch et al. (2002); Mai et al. (2002); Vigushin et al. (2002); Gottlicher et al. (2001); Jung (2001); Komatsu et al. (2001); Su et al. (2000).

### IV. Methods of Treating

Heart disease of some forms may curable and these are dealt with by treating the primary disease, such as anemia or thyrotoxicosis. Also curable are forms caused by anatomical problems, such as a heart valve defect. These defects can be surgically corrected. However, for the most common forms of heart failure—those due to damaged heart muscle—no known cure exists. Treating the symptoms of these diseases helps, and some treatments of the disease have been successful. The treatments attempt to improve patients' quality of life and length of survival through lifestyle change and drug therapy.

Patients can minimize the effects of heart failure by controlling the risk factors for heart disease, but even with lifestyle changes, most heart failure patients must take medication, many of whom receive two or more drugs.

Several types of drugs have proven useful in the treatment of heart failure: Diuretics help reduce the amount of fluid in the body and are useful for patients with fluid retention and hypertension; and digitalis can be used to increase the force of the heart's contractions, helping to improve circulation. Results of recent studies have placed more emphasis on the use of ACE inhibitors (Manoria and Manoria, 2003). Several large studies have indicated that ACE inhibitors improve survival among heart failure patients and may slow, or perhaps even prevent, the loss of heart pumping activity (for a review see De Feo et al., 2003; DiBianco, 2003).

Patients who cannot take ACE inhibitors may get a nitrate and/or a drug called hydralazine, each of which helps relax tension in blood vessels to improve blood flow (Ahmed, 2003).

Heart failure is almost always life-threatening. When drug therapy and lifestyle changes fail to control its symptoms, a heart transplant may be the only treatment option. However, candidates for transplantation often have to wait months or even years before a suitable donor heart is found. Recent studies indicate that some transplant candidates improve during this waiting period through drug treatment and other therapy, and can be removed from the transplant list (Conte et al., 1998).

Transplant candidates who do not improve sometimes need mechanical pumps, which are attached to the heart. Called left ventricular assist devices (LVADs), the machines take over part or virtually all of the heart's blood-pumping activity. However, current LVADs are not permanent solutions for heart failure but are considered bridges to transplantation.

As a final alternative, there is an experimental surgical procedure for severe heart failure available called cardiomyoplasty. (Dumcius et al., 2003) This procedure involves detaching one end of a muscle in the back, wrapping it around the heart, and then suturing the muscle to the heart. An implanted electric stimulator causes the back muscle to contract, pumping blood from the heart. To date, none of these treatments have been shown to cure heart failure, but can at least improve quality of life and extend life for those suffering this disease.

As with heart failure, there are no known cures to hypertrophy. Current medical management of cardiac hypertrophy, in the setting of a cardiovascular disorder includes the use of at least two types of drugs: inhibitors of the rennin-angiotensin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn & Bristow, 1996). Other pharmaceutical agents that have been disclosed for treatment of cardiac hypertrophy include angiotensin II receptor antagonists (U.S. Pat. No. 5,604,251) and neuropeptide Y antagonists (PCT Publication No. WO 98/33791).

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

As can be seen from the discussion above, there is a great need for a successful treatment approach to heart failure and hypertrophy. In one embodiment of the present invention, methods for the treatment of cardiac hypertrophy or heart failure utilizing modulators of MCIP-1-38 are provided. For the purposes of the present application, treatment comprises reducing one or more of the symptoms of heart failure or cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of modulators of MCIP-1-38 may prevent cardiac hypertrophy and its associated symptoms from arising.

A. Pharmaceutical Inhibitors

MCIP-1 has only recently been studied and MCIP-1-38 is newly described herein. As such, no modulators have been discovered aside from those described herein (and which shall be a subject of a later application). The inventors have discovered compounds that upregulate MCIP-1-38

B. Antisense Constructs

An alternative approach to upregulating MCIP-1-38 would be utilization of antisense technology. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit or promote gene transcription or translation or both within a host cell, either in vitro or in vivo, such as within a host animal, including a human subject. Promotion of gene transcription may lead to upregulation of MCIP-1-38, while inhibition of gene transcription could inhibit the transcription of a repressor gene that controls MCIP-1-38 expression. Thus, one of skill can easily envision ways in which antisense could be used to promote MCIP-1-38 expression.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

C. Ribozymes

Another general class of inhibitors is ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

D. RNAi

RNA interference (also referred to as “RNA-mediated interference” or RNAi) is another mechanism by which MCIP-1-38 expression could be modulated in a way similar to that of the antisense methodology. One can envision instances when inhibitory RNAs could be reduced or eliminated, leading to increased expression of MCIP-1-38. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Treatment regimens would vary depending on the clinical situation. However, long term maintenance would appear to be appropriate in most circumstances. It also may be desirable treat hypertrophy with modulators of MCIP-1-38 intermittently, such as within brief window during disease progression.

E. Antibodies

In certain aspects of the invention, antibodies may find use as modulators of MCIP-1-38 expression. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

For detection of MCIP-1-38 protein sequences, a diagnostic kit of the present invention comprises, in one or more containers, an anti-MCIP-1-38 antibody which optionally can be detectably labeled. In a different embodiment, the kit can comprise in a container, a labeled specific binding portion of an antibody. As used herein, the term detectable label refers to any label which provides directly or indirectly a detectable signal and includes, for example, enzymes, radiolabelled molecules, fluorescent molecules, particles, chemiluminesors, enzyme substrates or cofactors, enzyme inhibitors, or magnetic particles. Examples of enzymes useful as detectable labels in the present invention include alkaline phosphatase and horse radish peroxidase. A variety of methods are available for linking the detectable labels to proteins of interest and include for example the use of a bifunctional agent, such as, 4,4′-difluoro-3,3′-dinitro-phenylsulfone, for attaching an enzyme, for example, horse radish peroxidase, to a protein of interest. The attached enzyme is then allowed to react with a substrate yielding a reaction product which is detectable. The present invention provides a method for detecting an MCIP-1-38 protein in a patient sample, comprising, contacting the patient sample with an anti-MCIP-1-38 antibody under conditions such that immunospecific binding can occur, and detecting or measuring the amount of any immunospecific binding by the antibody. The method can be performed in situ in PMBCs, in cell lysate such as an ELISA, or cell lysate or purified protein in a western blot.

F. Combined Therapy

In another embodiment, it is envisioned to use a modulator of MCIP-1-38 in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using a modulator of MCIP-1-38 may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a modulator of MCIP-1-38, or the other agent will be desired. In this regard, various combinations may be employed. By way of illustration, where the modulator of MCIP-1-38 is “A” and the other agent is “B,” the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated.

G. Adjunct Therapeutic Agents

Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In addition, it should be noted that any of the following may be used to develop new sets of cardiac therapy target genes as β-blockers were used in the present examples (see below). While it is expected that many of these genes may overlap, new gene targets likely can be developed.

1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of atherosclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog; a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thyroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, b-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of atherosclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and warfarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemorrhage or an increased likelyhood of hemorrhaging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocanide (tonocard) and mexiletine (mexitil). Non-limiting examples of Class IC antiarrhythmic agents include encamide (enkaid) and flecainide (tambocor).

b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a miscellaneous calcium channel blocker such as bencyclane, etafenone, magnesium, mibefradil or perhexiline. In certain embodiments a calcium channel blocker comprises a long-acting dihydropyridine (amlodipine) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherally acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimeylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, g aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

8. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine administration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.

c. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

H. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

I. Drug Formulations and Routes for Administration to Patients

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures

The previously mentioned formulations are all contemplated for treating patients suffering from heart failure or hypertrophy.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

### V. Screening Methods

The present invention further comprises methods for identifying modulators of MCIP-1-38 in cardiac cells that are useful in the prevention or treatment or reversal of cardiac hypertrophy or heart failure. These assays may comprise random screening of large libraries of candidate substances; alternatively, the assays may be used to focus on particular classes of compounds selected with an eye towards structural attributes that are believed to make them more likely to enhance the function or activity or expression or stability of MCIP-1-38.

To identify a modulator of MCIP-1-38, one generally will determine the expression of MCIP-1-38 in the presence and absence of the candidate substance. For example, a method generally comprises:
(a) providing a cell;
(b) contacting said cell with a candidate modulator; and
(c) measuring expression (or another parameter of MCIP-1-38 activity) of MCIP-1-38 in said cell;
   wherein an increase in expression (by RNA or protein) in the cell, as compared to an untreated cell, identifies the candidate modulator as a modulator of MCIP-1-38 (increased expression again may be increased RNA or protein expression, it may be increased stability or potency of MCIP-1-38, and it may be due to an indirect effect on another gene or gene product causing the increased expression of MCIP-1-38).

Assays also may be conducted in isolated cells, organs, or in living organisms.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate” or “candidate substance” refers to any molecule that may potentially alter or modulate the activity, stability, potency, efficacy, or cellular functions of MCIP-1-38. The candidate substance may be a protein or fragment thereof, a small molecule, or even a nucleic acid. It may prove to be the case that the most useful pharmacological compounds will be compounds that are discovered through high-throughput screens of large compound libraries. Using lead compounds and the application of generally accepted good practices of medicinal chemistry to help develop improved compounds is known as “rational drug design” and includes not only comparisons with know inhibitors and activators, but predictions relating to the structure of target molecules, and especially in this later case the term “structure-based drug design” is sometimes used to describe the process.

The goal of rational drug design is to produce compounds with improved, biologically activity. By creating such and developing structure-activity relationships (SAR or QSAR) it is possible to invent drugs which are more active or stable than the starting molecules, which have different susceptibility to metabolism, or which may affect the function of various other molecules. Part of this process is commonly referred to as ADME or ADMET, and one of skill in the art will understand that herein are described attempts to improve the ADME or ADMET properties of a compound or lead series of compounds (administration, distribution, metabolism, elimination and toxicology).

In one approach, one would generate a three-dimensional structure for a target molecule, or a fragment thereof. This could be accomplished by x-ray crystallography, computer modeling, or by a combination of both approaches.

On the other hand, one may simply acquire, from various commercial sources, small molecule libraries of compounds that are believed to meet the basic criteria for useful drugs in an effort to discover through screening the identification of useful compounds. Screening of such libraries, including combinatorially-generated libraries (e.g., small molecule or peptide libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be a peptide, polypeptide, protein, polynucleotide, small molecule or any other compounds that may be designed through rational drug design starting from known compounds.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other similar compounds may be formulated to mimic the key portions of the structure of the modulators. Such compounds, which may include peptidomimetics of peptide modulators, may be used in the same manner as the initial modulators.

B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small peptide test compounds are synthesized on a solid substrate, such as plastic pins or some other surface. Such peptides could be rapidly screening for their ability to enhance MCIP-1-38 expression.

C. In Cyto Assays

The present invention also contemplates the screening of compounds for their ability to upregulate MCIP-1-38 in cells. Various cell lines can be utilized for such screening assays, including cells specifically engineered for this purpose.

D. In Vivo Assays

In vivo assays involve the use of various animal models of heart disease, including transgenic animals, that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria, including but not limited to. Also, measuring toxicity and dose response can be performed in animals in a more meaningful fashion than in in vitro or in cyto assays.

### VI. Vectors for Cloning, Gene Transfer and Expression

Within certain embodiments, expression vectors are employed to express various products including MCIP-1-38, antisense molecules, ribozymes or interfering RNAs. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the native MCIP-1 promoter will be employed to drive expression of either the corresponding gene, a heterologous MCIP-1 gene, a screenable or selectable marker gene, or any other gene of interest.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; |
| | Grosschedl et al., 1985; Atchinson et al., 1986, |
| | 1987; Imler et al., 1987; Weinberger et al., |
| | 1984; Kiledjian et al., 1988; Porton et al.; |
| | 1990 |
| Immunoglobulin Light Chain | Queen et al., 1983; Picard et al., 1984 |
| T-Cell Receptor | Luria et al., 1987; Winoto et al., 1989; |
| | Redondo et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; |
| | Johnson et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor (PDGF) | Pech et al., 1989 |
| Duchenne Muscular Dystrophy | Klamut et al., 1990 |
| SV40 | Banerji et al., 1981; Moreau et al., 1981; |
| | Sleigh et al., 1985; Firak et al., 1986; Herr et |
| | al., 1986; Imbra et al., 1986; Kadesch et al., |
| | 1986; Wang et al., 1986; Ondek et al., 1987; |
| | Kuhl et al., 1987; Schaffner et al., 1988 |
| Polyoma | Swartzendruber et al., 1975; Vasseur et al., |
| | 1980; Katinka et al., 1980, 1981; Tyndell et |
| | al., 1981; Dandolo et al., 1983; de Villiers et |
| | al., 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Choi et al., 1988; |
| | Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987 |
| Hepatitis B Virus | Bulla et al., 1986; Jameel et al., 1986; Shaul et |
| | al., 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; |
| | Takebe et al., 1988; Rosen et al., 1988; |
| | Berkhout et al., 1989; Laspia et al., 1989; |
| | Sharp et al., 1989; Braddock et al., 1989 |
| Cytomegalovirus (CMV) | Weber et al., 1984; Boshart et al., 1985; |
| | Foecking et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; |
| | | Stuart et al., 1985; |
| | | Imagawa et al., 1987, |
| | | Karin et al., 1987; |
| | | Angel et al., 1987b; |
| | | McNeall et al., 1989 |
| MMTV (mouse | Glucocorticoids | Huang et al., 1981; |
| mammary tumor virus) | | Lee et al., 1981; |
| | | Majors et al., 1983; |
| | | Chandler et al., 1983; |
| | | Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, |
| | Antigen | 1990a, 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Of particular interest are muscle specific promoters, and more particularly, cardiac specific promoters. These include the myosin light chain-2 promoter (Franz et al., 1994; Kelly et al., 1995), the alpha actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the alpha7 integrin promoter (Ziober & Kramer, 1996), the brain natriuretic peptide promoter (LaPointe et al., 1995) and the alpha B-crystallin/small heat shock protein promoter (Gopal-Srivastava, R., 1995), alpha myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g, 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpes viruses may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, E. P. App. 273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### VII. Preparing Antibodies to MCIP-1-38

In yet another aspect, the present invention contemplates the use of antibodies that may bind to MCIP-1-38 or some associated factor or protein involved in the disease process mediated by MCIP-1-38. An antibody can be a polyclonal or a monoclonal antibody, it can be humanized, single chain, or even an Fab fragment. In a preferred embodiment, an antibody is a monoclonal antibody. Means for preparing and characterizing antibodies are well known in the art (see Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to MCIP-1-38 antigen epitopes.

In general, both polyclonal, monoclonal, and single-chain antibodies against MCIP-1 may be used in a variety of embodiments. A particularly useful application of such antibodies is in purifying native or recombinant MCIP-1, for example, using an antibody affinity column. The operation of all accepted immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified PKD protein, polypeptide or peptide or cell expressing high levels of PKD. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 41, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### VIII. Definitions

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rates and the like including laboratory findings associated with heart failure.

The term “treatment” or grammatical equivalents encompasses the prevention, improvement and/or reversal of symptoms of a specific disease, disorder, syndrome or state (i.e., improving the ability of the heart to pump blood in a heart failure setting). Improvement in the physiologic function of the heart may be assessed using any of the measurements described herein (e.g., measurement of ejection fraction, fractional shortening, left ventricular internal dimension, heart rate, etc.), as well as any effect upon the animal's survival. A compound which causes an improvement in any parameter associated with a specific disease used in the screening methods of the instant invention may thereby be identified as a therapeutic compound.

The terms “compound” and “chemical agent” may refer to any chemical entity, pharmaceutical, drug, protein, antibody, nucleic acid and the like that can be used to treat or prevent a disease, illness, sickness, or disorder of bodily function. Compounds and chemical agents comprise both known and potential therapeutic compounds. A compound or chemical agent can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “cardiac hypertrophy” refers to the process in which adult cardiac myocytes respond to a wide variety of pathophysiological, chemical, external and biological stresses through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the term “modulator” refers to any agent which is capable of altering the expression, stability, activity, efficacy, or potency of MCIP-1-38. Modulators may include proteins, nucleic acids, carbohydrates, peptides, small molecules, antibodies, or any other molecule(s) which binds or interacts with a cellular or intracellular receptor, molecule, and/or pathway of interest. Modulators need not act directly on MCIP-1-38 protein or the gene locus, but may cause an upregulation of expression or activity or function (at the RNA or protein level) indirectly, via an effect on some other gene or protein that leads to upregulation or increased expression of MCIP-1-38.

As used herein, the term “modulate” refers to a change or an alteration in a biological or chemical activity. Modulation may be an increase or a decrease in protein activity, a change in kinase activity, a change in binding characteristics, or any other change in the biological, functional, or immunological properties associated with the activity of a protein or other structure of interest.

As used herein, the term “select” or “selection” in the context of a modulator will be understood to mean making a choice between known or experimental compounds and agents.

As used herein, the term “small molecule” refers to an organic molecule or its salt(s), usually having a molecular weight less than 1000 Daltons.

### VIII. Examples

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1

### Materials and Methods

MCIP-1 polyclonal antibody production. A peptide corresponding to the carboxy-terminus of murine MCIP-1 protein (accession #AAF63486; CRPEYTPIHLS) was synthesized (Sigma Genosys), incorporating an amino-terminal cysteine residue to facilitate conjugation to keyhole limpet hemocyanin (KLH) carrier. Rabbits were immunized with KLH-conjugated peptide according to standard polyclonal antibody production protocols (Lampire Biological Laboratories).

NRVM culture. For preparations of neonatal rat ventricular myocytes (NRVMs), hearts were removed from 10-20 newborn (1-2 days old) Sprague-Dawley rats. Isolated ventricles were pooled, minced and dispersed by three 20-minute incubations at 37° C. in Ads buffer (116 mM NaCl, 20 mM HEPES, 10 mM NaH2PO4, 5.5 mM glucose, 5 mM KCl, 0.8 nM MgSO4, pH 7.4) containing collagenase Type II (65 units/ml, Worthington) and pancreatin (0.6 mg/ml, GibcoBRL). Dispersed cells were applied to a discontinuous gradient of 40.5% and 58.5% (v/v) Percoll (Amersham Biosciences), centrifuged, and myocytes collected from the interface layer. Myocyte preparations were pre-plated in Dulbecco's modified Eagle's medium (DMEM, Cellgro), supplemented with 10% (v/v) fetal bovine serum (FBS, HyClone), 4 mM L-glutamine and 1% penicillin/streptomycin for 1 hour at 37° C. to reduce fibroblast contamination, then plated at a density of 2.5′105 cells per well on 6-well tissue culture plates (or 10,000 cells/well on 96-well tissue culture plates) coated with a 0.2% (w/v) gelatin solution. After 24 hours in culture, myocyte preparations were transferred to serum-free maintenance medium (DMEM supplemented with 0.1% (v/v) Nutridoma (Roche), L-glutamine and penicillin/streptomycin). For infection with calcineurin adenovirus, NRVM were exposed to adenovirus at a multiplicity of infection (MOI) of 25 for 48 h prior to analysis. Where indicated, NRVM were treated with phenylephrine (20 mM, Sigma) for 48 h.

Western Blots. For protein sample preparation, cultured cells were lysed in extraction buffer (50 mM Tris, pH 7.5, 150 mM NaCl, 1% Triton X-100, 0.5% deoxycholic acid, 0.1% SDS) supplemented with protease inhibitors (1 mM AEBSF, 10 mg/ml aprotinin, 0.1 mM leupeptin, 2 mM EDTA). Tissue samples were ground under liquid nitrogen and solubilized in extraction buffer containing protease inhibitors. Homogenates were centrifuged 10 min at 4° C. at 16,000 g and supernatants recovered. Protein concentrations were determined by the bicinchoninic acid method (BCA Protein Assay, Pierce) with bovine serum albumin as a standard. Equivalent quantities of protein samples (10 mg/lane) were denatured in Laemmli buffer and resolved on Tris-glycine SDS-PAGE gels (4-20% acrylamide gradient, Invitrogen). Resolved proteins were transferred to nitrocellulose membranes, blocked in 5% nonfat dry milk, and probed with rabbit anti-MCIP-1 polyclonal primary antibody (diluted 1:5000 in TBST; 50 mM Tris, pH 7.5, 150 mM NaCl, 0.1% Tween-20) supplemented with 5% nonfat dry milk. Membranes were washed, probed with a goat anti-rabbit horseradish peroxidase-conjugated secondary antibody (Southern Biotechnology Associates), and processed for enhanced chemiluminescence (SuperSignal reagent, Pierce). To verify equivalent protein loading, membranes were subsequently reprobed with a polyclonal rabbit antibody to the housekeeping gene IP90-calnexin. Densitometric analysis of immunoreactive band images was performed using a ChemiImager (Alpha Innotech).

Human MCIP-1 affinity purification and peptide mass fingerprinting. Ten grams of human left ventricle were homogenized by Polytron in 50 ml TBS supplemented with protease inhibitors (1 mM AEBSF, 10 mg/mil aprotinin, 0.1 mM leupeptin, 2 mM EDTA). A detergent mixture of Triton X-100 (1% final), deoxycholic acid (0.5% final) and SDS (0.1% final) was added, and the homogenate was centrifuged 10 min at 4° C. at 16,000′ g. The supernatant was recovered, denatured by addition of SDS (1% final) and incubation at 95° C. for 10 min, then re-centrifuged 10 min at 16,000′ g. The supernatant was loaded into dialysis cassettes (Vendor) and dialyzed overnight at 4° C. in 1 liter TBS+0.1% SDS. Fifty microliters of anti-MCIP1 antiserum was added to the dialyzed sample, and allowed to incubate overnight at 4° C. with continuous mixing. Protein G sepharose beads (250 ml packed bead volume, Vendor) were then added, followed by overnight incubation at 4° C. with continuous mixing. The beads were washed three times in 10 ml cold TBS+1% Triton X-100, and bound protein recovered by resuspending washed beads in 1 ml 0.5 M acetic acid (pH 3.0) for 1 minute. The eluate was neutralized with 250 ml 2M Tris base, then resolved by SDS-PAGE and colloidal Coomassie staining.

The stained band corresponding to the MCIP-1 Western blot band at 38 kDa was excised from the gel and processed for peptide mass fingerprinting using matrix assisted laser desorption/ionization (MALDI) time of flight (TOF) mass spectrometry. The gel piece was placed in a 0.3 ml glass vial and washed twice with 50% acetonitrile/25 mM ammonium bicarbonate and once with acetonitrile. The gel piece was dried in a speedvac and rehydrated with 10 ml of 50 mM ammonium bicarbonate, pH 8, containing 200 ng of sequencing grade trypsin (Promega) on ice for 20 minutes. The hydrated gel piece was incubated at 37oC overnight. Tryptic peptides were extracted with 200 ml of 50% acetonitrile/0.1% TFA. The gel piece was removed and the peptide extract was taken to dryness in a speedvac. The peptides were resolubilized overnight in 20 ml of 0.1% TFA and bound to a ZipTip, C18, 0.6 ml bed volume (Millipore) that had been wetted with 50% acetonitrile/0.1% TFA and equilibrated with 0.1% TFA. The ZipTip was washed three times with 0.1% TFA. A 2 ml aliquot of 80% acetonitrile/0.1% TFA was placed into a clean 0.3 ml glass vial and used to elute the peptides. The 2 ml of eluted peptide were spotted onto a stainless steel MALDI-TOF mass spectrometry plate. A 1 ml aliquot of matrix solution was immediately spotted on top. The matrix solution consisted of recrystallized a-cyano-4-hydroxy cinnamic acid (CHCA) dissolved in 80% ACN/0.1% TFA at a concentration of 10 mg/ml. The peptide and matrix mixture was allowed to dry and subjected to MALDI-TOF MS. Spectra were acquired on a Voyager-DE PRO spectrometer (PerSeptive Biosystems) operating in reflector mode. Spectra were internally calibrated on the trypsin autolytic peptide masses. Observed peptide masses were searched against the NCBInr database at a tolerance of 0.05 Da using the Mascot program (Matrix Science).

In vivo cardiac hypertrophy model: trans-thoracic aortic banding (TAB). For chronic left thoracotomy and aortic ligation, male Sprague-Dawley rats (Harlan, Indianapolis, Ind.; 8-9 weeks of age, 200-225 g) were anesthetized with 5% isoflurane (v/v 100% O2), intubated and maintained at 2.0% isoflurane with positive pressure ventilation. A left thoracotomy through the third intercostal space was performed and the descending thoracic aorta, 3-4 mm cranial to the intersection of the aorta and azygous vein was isolated. A segment of 5-0 silk suture was then positioned around the isolated aorta to function as a ligature. A blunted hypodermic needle (gauge determined by weight) was placed between the aorta and the suture to prevent complete aortic occlusion when the suture was tied. When tying was completed, the needle was removed from between the aorta and ligature, re-establishing flow through the vessel. The thorax was then closed and the pneumothorax evacuated. After 7 days of recovery, animals were sacrificed and left ventricular tissue processed for Western blot analysis as described above. Average heart weight to body weight ratios in banded versus sham-operated rats increased 22% at 1 week (data not shown).

Human LV panel. Twelve human non-failing ventricular samples were obtained from organ donors whose hearts were unsuitable for donation due to blood type or size incompatibilities (5 male, 7 female; mean age 48.5 yr). Twelve end-stage failing ventricular samples were obtained from individuals who underwent heart transplantation due to idiopathic dilated cardiomyopathy (6 male, 6 female; mean age 49.3 yr). Tissue samples were taken immediately upon explantation and rapidly frozen in liquid nitrogen.

Hypertrophy and toxicity assays. Primary hypertrophy endpoints for NRVM included quantitation of ANF secretion, total cellular protein and cell volume. ANF in media supernatants was quantitated by competitive ELISA using a monoclonal anti-ANF antibody (Biodesign) and a biotinylated ANF peptide (Phoenix Peptide). Total cellular protein was quantitated by standard Coomassie dye-binding assay; cells were lysed in protein assay reagent (BioRad) and absorbance at A595 was measured after 1 hour. For cell volume measurements, NRVM cultured in 6-well dishes were harvested by treatment with trypsin (Cellgro). After recovery by centrifugation, cell pellets were washed in PBS, resuspended in 10 ml IsoFlow electrolyte solution (Beckman-Coulter) and analyzed with a Z2 Coulter Particle Counter and Size Analyzer (Beckman-Coulter). Cytotoxicity was measured by quantitation of intracellular ATP (CellTiter-Glo Kit, Promega).

Myosin heavy chain protein quantitation by cytoblot. NRVM were plated overnight in 96-well plates. The next day, medium was replaced with serum-free maintenance medium for 4 hours, and test compounds added. Forty eight hours later, wells were washed twice with 100 ml/well PBS, aspirating between washes. Cells were fixed by adding 100 ml/well methanol for 30 min. Methanol was aspirated and wells washed twice with 100 ml/well PBS. Next, 100 ml/well blocking solution (PBS+1% BSA) was added for 1 hr at room temperature. Blocking solution was aspirated and 50 ml/well primary antibody solution added (alpha or beta myosin heavy chain hybridoma supernatant +1% BSA) for 1 hr at room temperature. Primary antibody solution was removed and wells washed three times with 100 ml/well PBS+1% BSA. Wash was aspirated and 50 ml/well secondary antibody solution added (1:500 dilution of goat anti-rabbit HRP conjugate in PBS+1% BSA; Southern Biotech #4050-05) for 1 hr at room temperature. Secondary antibody solution was removed and wells washed three times with 100 ml/well PBS. Wash was aspirated and 50 ml/well luminol solution added (Pierce #34080). Plates were read in a 96-well luminometer (Packard Fusion).

RNA dot blot analysis. NRVM were plated overnight in 10 cm plates. The next day, medium was replaced with serum-free maintenance medium for 4 hours, and test compounds added. Forty eight hours later, cells were washed twice with 100 ml/well PBS, aspirating between washes. NRVM were lysed and total RNA extracted as per manufacturer's recommendations (Tri reagent, Sigma). Using a vacuum manifold, twenty micrograms of denatured total RNA per sample were applied to a nylon membrane (Nytran, Schleicher and Schuell) and crosslinked using a UV transilluminator. Membranes were hybridized overnight with 32P-labeled oligonucleotide probes to atrial natriuretic factor (ANF), alpha skeletal actin or a control probe to GAPDH. Membranes were hybridized and washed as per manufacturer's instructions (Ultrahyb Oligo Hybridization Buffer, Ambion Inc.). Autoradiography was performed by exposing washed membranes to X-ray film overnight.

Rat peripheral blood mononuclear cell (PBMC) preparation. Heparinized whole blood was layered on top of a Ficoll cushion in a centrifuge tube as per manufacturer's instructions (Ficoll Paque PLUS, Amersham Biosciences). Tubes were centrifuged at room temperature in a swinging bucket rotor for 45 minutes at 1000×g. Cells from the buffy coat layer were collected, washed in PBS (minus calcium and magnesium), and centrifuged at 4 C for 15 minutes at 430×g. The resulting PBMC pellet was then processed for MCIP1 Western blot analysis as previously described.

### B. Results

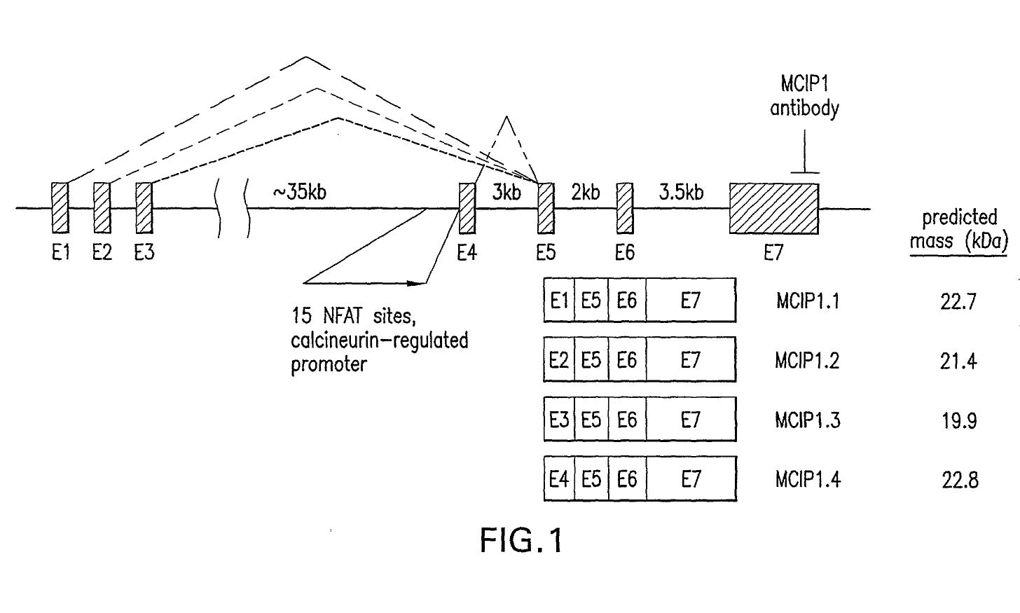

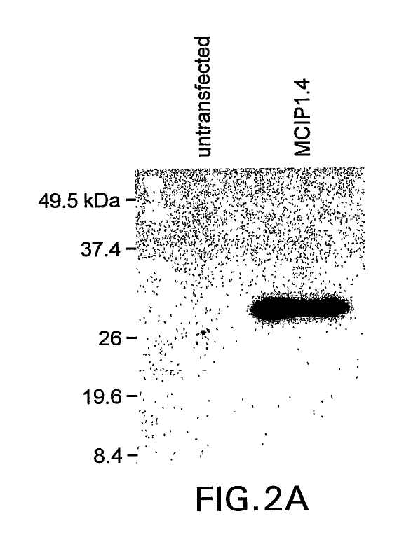

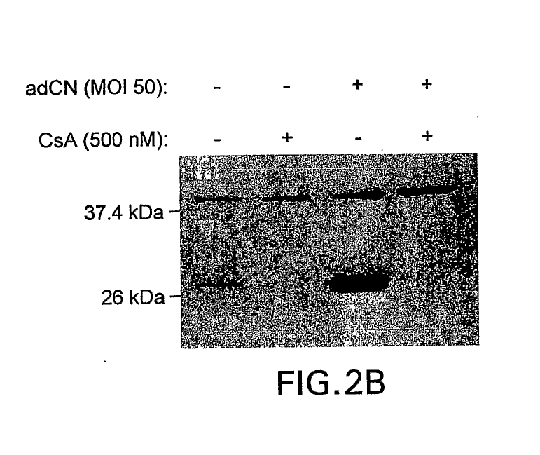

Identification of a 38 kDa protein (MCIP-1-38) encoded by the MCIP-1 locus. As shown in FIG. 1, the four known MCIP-1 transcripts utilize alternative promoters and possess unique first exons, but share common exons 5, 6 and 7 (adapted from Rothermel et al., 2003). The molecular masses of the proteins encoded by all MCIP-1 transcripts are predicted to be approximately 23 kDa or less. The inventors created an MCIP-1-specific polyclonal antibody by immunizing rabbits with a synthetic peptide corresponding to the 10 C-terminal peptides present in all MCIP-1 forms (described in Materials and Methods). The MCIP-1 antibody recognized a single recombinant protein band in mammalian cells transfected with a vector expressing an MCIP-1.4 cDNA, confirming that the protein product of the MCIP-1.4 transcript is approximately 28 kDa in size (FIG. 2A). In Western blots of protein isolated from cultured rat cardiomyocytes, the MCIP-1 antibody also recognized an approximately 28 kDa endogenous calcineurin-inducible protein (FIG. 2B). Exposing myocytes to the calcineurin inhibitor cyclosporine A (CsA) suppressed expression of the endogenous 28 kDa protein, consistent with this protein being the product of the calcineurin-regulated MCIP-1.4 transcript. The inventors also observed an approximately 38 kDa higher molecular weight immunoreactive protein, significantly larger than the predicted molecular masses of the known MCIP-1 transcripts. The endogenous 38 kDa protein was not induced by exogenous calcineurin or suppressed by exposing myocytes to CsA, suggesting that 38 kDa protein expression is regulated independently of calcineurin activity.

Since the 38 kDa MCIP1 protein was substantially larger than the predicted molecular masses of currently described MCIP-1 splice variants, the inventors independently verified the identity of the 38 kDa protein by peptide mass fingerprinting. Using the MCIP-1 antibody as an affinity reagent, a highly enriched fraction of 38 kDa protein was purified from human left ventricular tissue, digested with trypsin and subjected to MALDI-TOF mass spectrometry. The masses of the resultant tryptic peptides were searched against the NCBInr database using the Mascot program (Matrix Science). Seven peptides matched with a statistically significant score to MCIP-1 (Table I), confirming that the 38 kDa protein is a product of the MCIP-1 locus. Interestingly, while the inventors recovered peptides corresponding to common exons 5-7, no splice variant-specific peptides were identified from the variable amino terminus (exons 1, 2, 3 or 4). Since peptide mass fingerprinting can only match peptides to known sequences reported in the public protein databases, this result is consistent with the interpretation that the 38 kDa protein encodes a form of MCIP-1 with a unique N-terminus.

TABLE 1

| Observed | | | | |
|---|---|---|---|---|
| Mass | Theoretical Mass | Delta | Sequence | MCIP1 Exon |
| 798.40 | 798.42 | 0.02 | FESLR | 5 |
| 1409.65 | 1409.68 | 0.03 | INFSNPFSAADAR | 5 |
| 638.38 | 638.40 | 0.02 | LQLHK | 5 |
| 694.34 | 694.38 | 0.04 | TEFLGK | 5 |
| 600.34 | 600.34 | 0.00 | LGPGEK | 6/7 |
| 630.38 | 630.39 | 0.01 | IIQTR | 7 |
| 1212.65 | 1212.64 | 0.01 | RPEYTPIHLS | 7 |

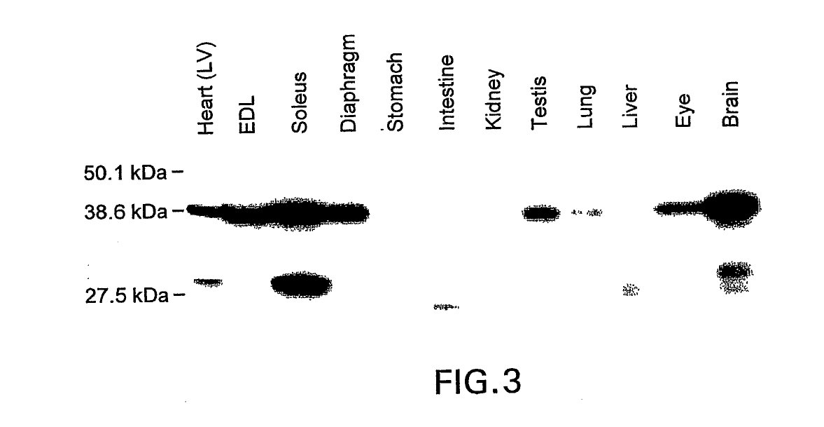

Tissue distribution of endogenous MCIP-1-38 protein. The inventors examined the expression pattern of endogenous MCIP-1 protein by Western analysis of a rat tissue panel (FIG. 3). Expression of 28 kDa MCIP-1 protein was most abundant in heart, soleus and brain; these results are consistent with previous reports of the distribution of MCIP-1.4 transcript. MCIP-1-38 protein exhibited a broader tissue distribution, comprising the major expressed form of MCIP1 in the heart, EDL, diaphragm, testis, lung, eye and brain.

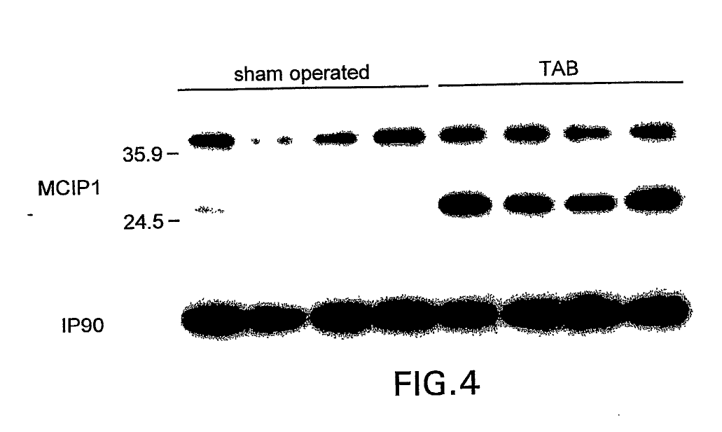

MCIP-1-38 protein expression in an in vivo model of cardiac hypertrophy. The inventors performed Western blots on left ventricular tissue obtained from a physiologic model of cardiac hypertrophy in the rat. Thoracic aortic banding (TAB) produces a potent physiologic stimulus for hypertrophy (pressure overload), which was associated with significant induction of calcineurin-regulated 28 kDa MCIP-1.4 protein expression (FIG. 4); this result is consistent with previously reported observations of increased MCIP-1.4 transcript expression with pressure overload. In contrast, MCIP-1-38 protein levels were essentially unchanged under conditions of pressure overload, suggesting that MCIP-1-38 expression is regulated independently from the calcineurin-inducible 28 kDa form.

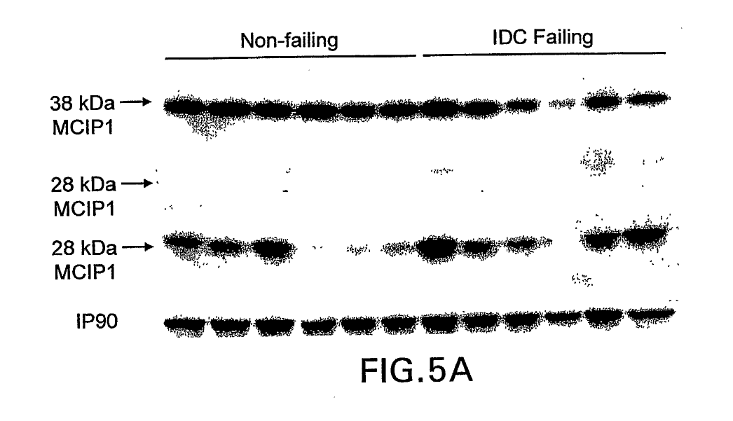

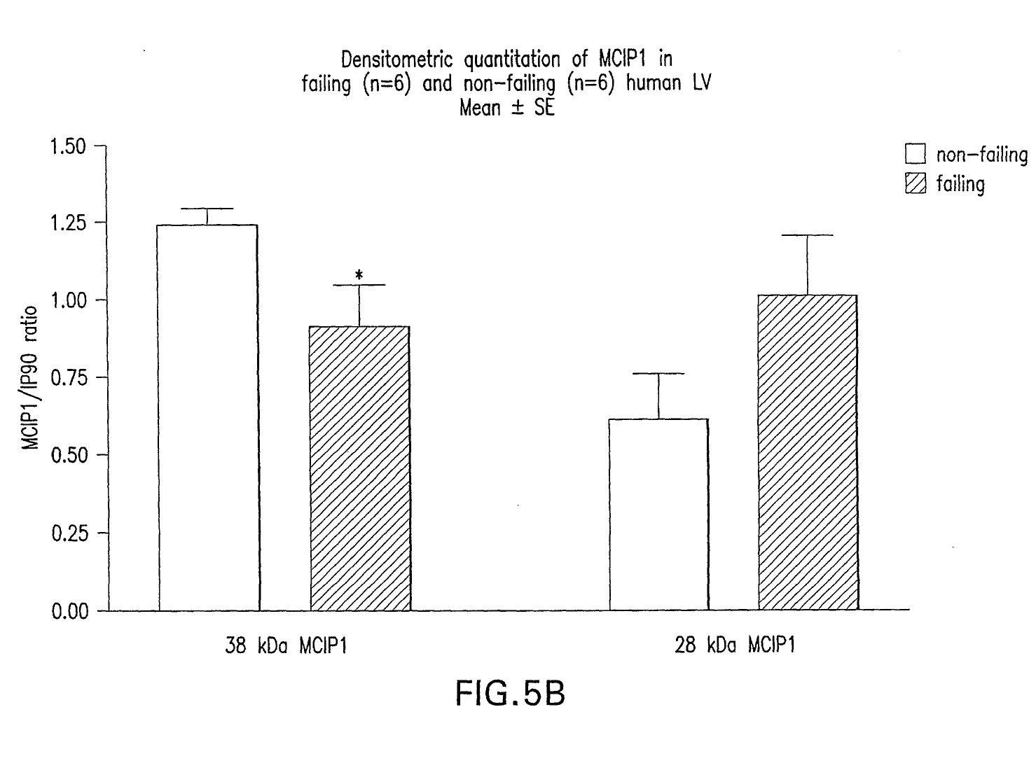

MCIP-1-38 comprises the predominant form of MCIP1 in the adult human left ventricle. Western blots of protein isolated from normal (non-failing) and failing (idiopathic dilated cardiomyopathy) human left ventricles revealed that the human heart expresses almost exclusively MCIP-1-38 protein (FIG. 5A). Densitometric analysis of MCIP-1-immunoreactive bands showed a trend towards decreased MCIP-1-38 and increased 28 kDa MCIP-1 in the failing human heart (FIG. 5B).

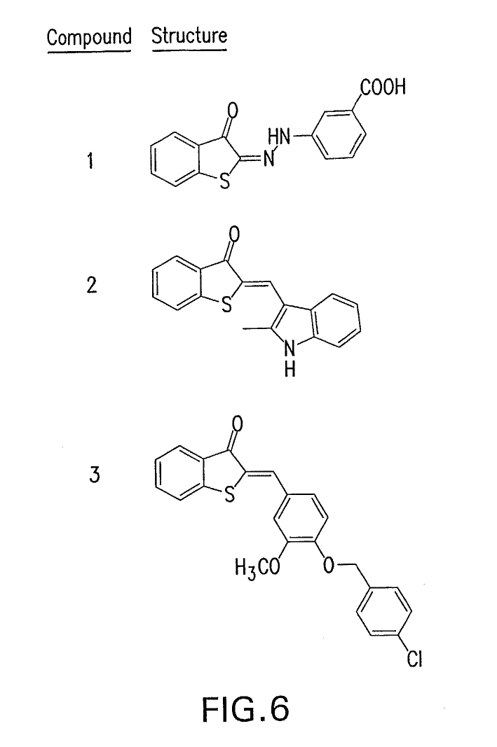

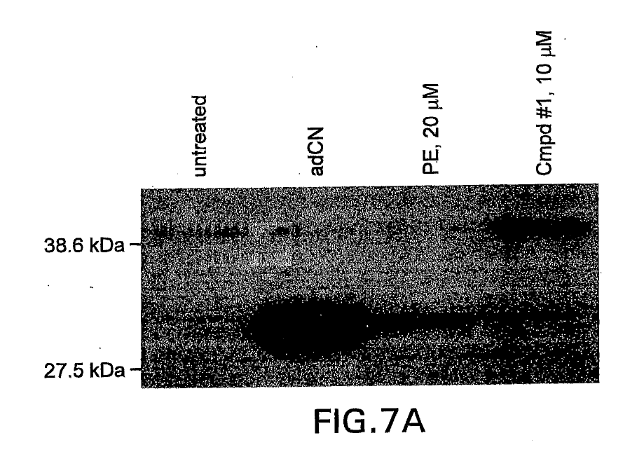

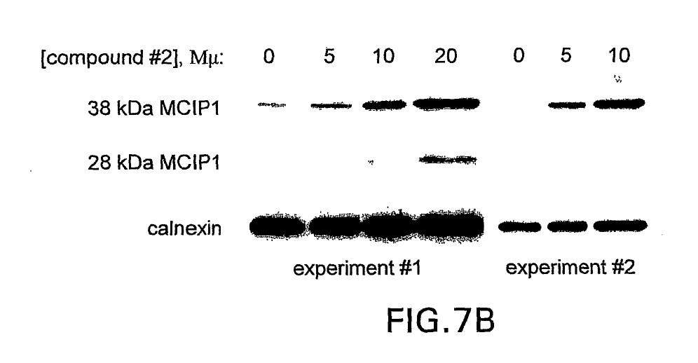

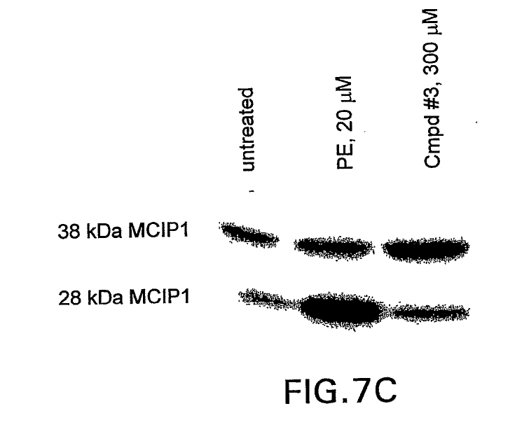

Small molecule enhancers of MCIP-1-38 expression. The inventors described three structurally related compounds that were found to selectively induce expression of endogenous MCIP-1-38 in cultured cardiac cells. Structures for the three compounds are shown in FIG. 6. As shown in FIG. 7A, cardiac myocytes exposed to compound #1 selectively increased expression of MCIP-1-38 protein. In contrast, pro-hypertrophic stimuli such as exogenous calcineurin or the α-adrenergic agonist phenylephrine (PE) selectively increased expression of calcineurin-regulated 28 kDa MCIP1 protein. Similar results are shown for compounds #2 and #3 (FIGS. 7B and 7C).

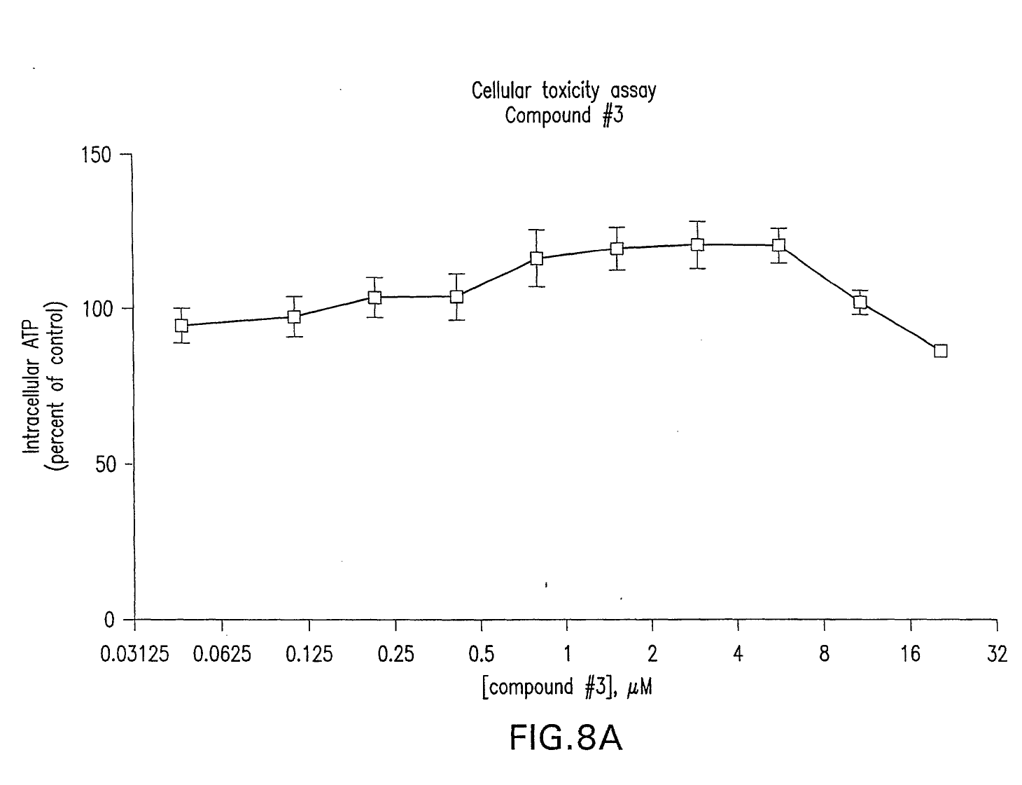

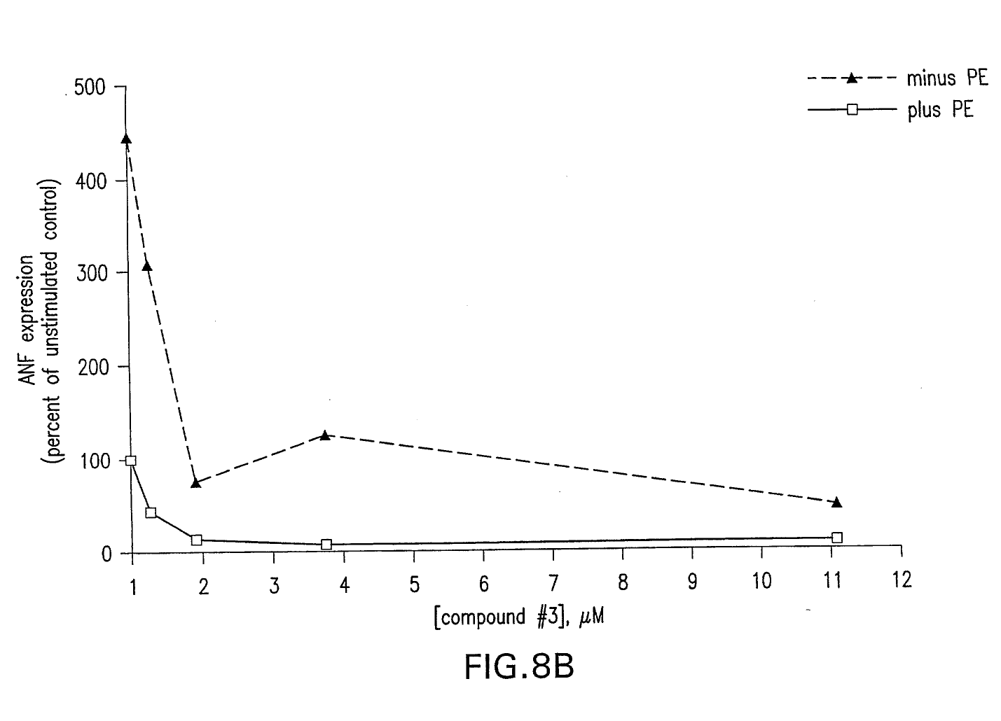

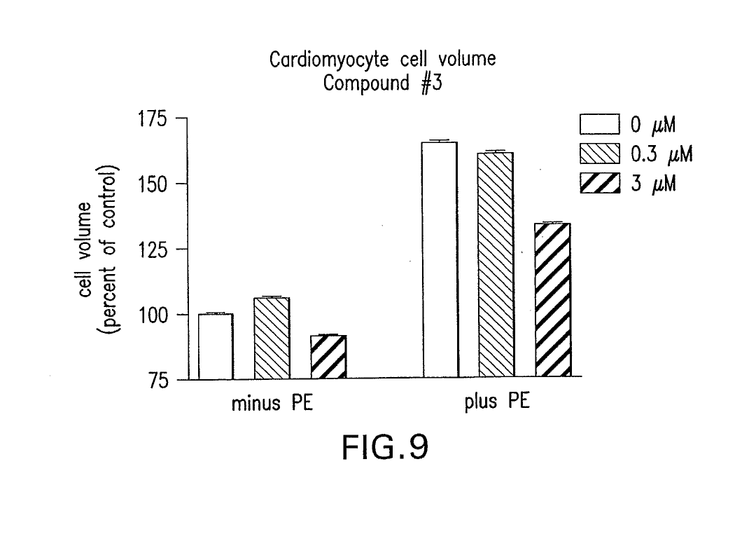

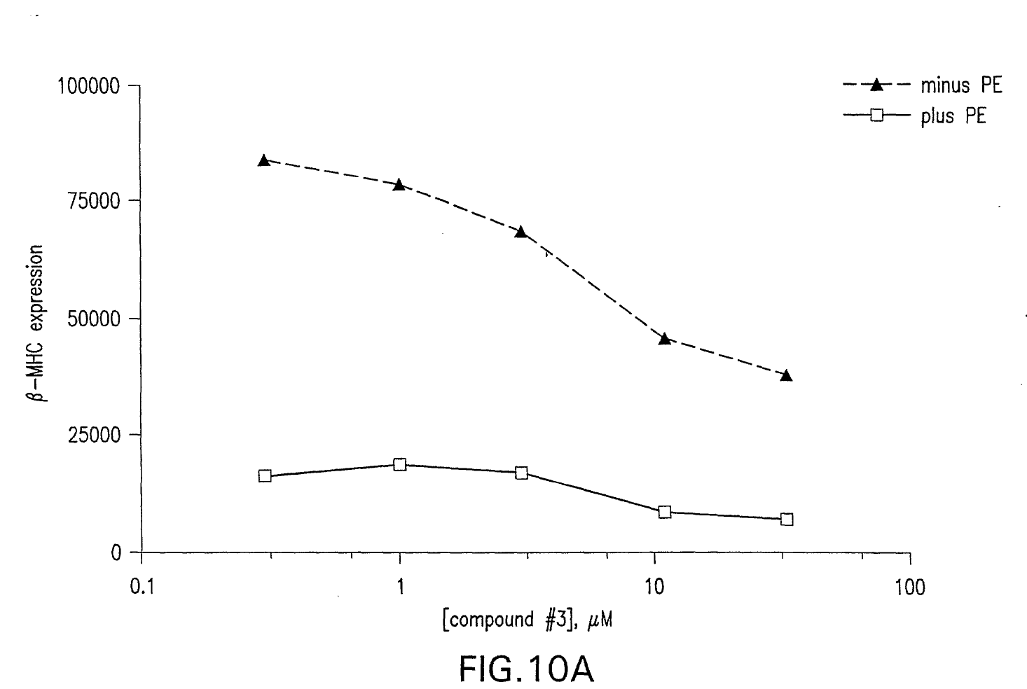

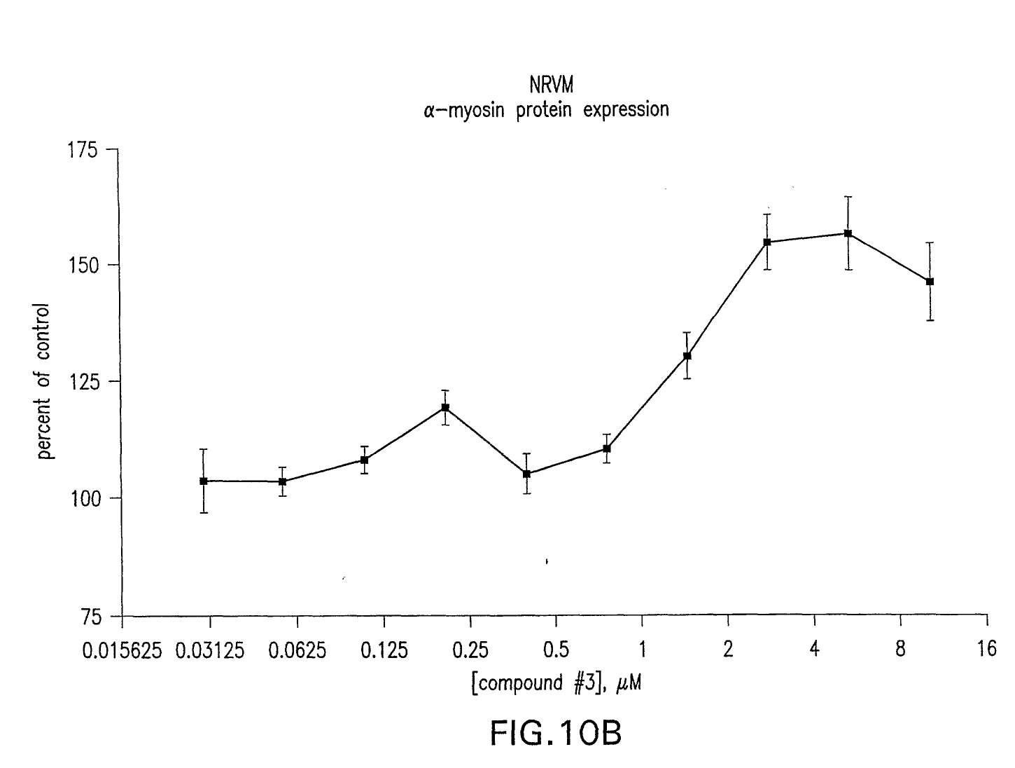

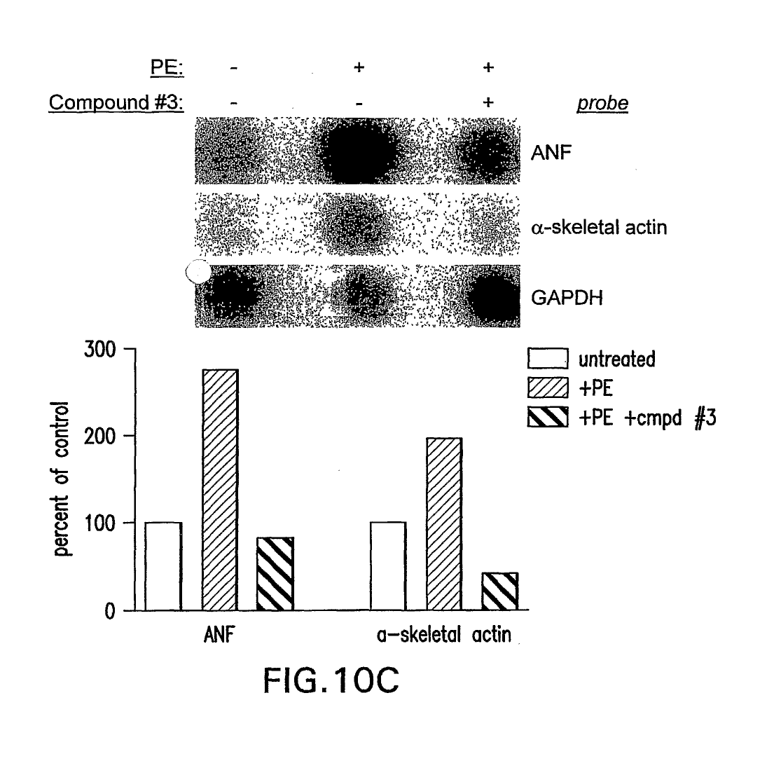

Small molecule-induced MCIP-1-38 expression correlates with reduced cardiomyocyte hypertrophy in vitro. The inventors demonstrated that at non-toxic concentrations (FIG. 8A), compound #3 effectively suppressed a variety of hypertrophic responses induced by exposure to the α-adrenergic agonist phenylephrine. The compound reduced secretion of atrial natriuretic factor (FIG. 8B) and attenuated the increase in cell volume associated with cardiomyocyte hypertrophy (FIG. 9). Compound #3 also normalized expression of fetal genes associated with cardiac hypertrophy. In cultured cardiomyocytes, compound #3 suppressed PE-dependent induction of fetal β-myosin heavy chain protein (FIG. 10A) and increased expression of the adult myosin isoform, α-myosin (FIG. 10B). Normalization of fetal gene expression by compound #3 likely occurs at the transcriptional level, since RNA dot blot experiments confirmed that PE-treated cardiomyocytes exposed to compound #3 expressed reduced mRNA levels of ANF and another standard marker of cardiac hypertrophy, α-skeletal actin (FIG. 10C).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. References

The following references, to the extent that they provide exemplary procedural or other details supplementary to those set forth herein, are specifically incorporated herein by reference.
Ahmed, A., J. Am. Geriatr. Soc., 51(1):123-126 (2003).
Aus. Pat. No. 6,794,700.
Aus. Pat. No. 9,013,101.
Aus. Pat. No. 9,013,201.
Aus. Pat. No. 9,013,401.
Baichwal and Sugden, Gene Transfer, Kucherlapati R, ed., New York, Plenum Press, pp. 117-148, 1986.
Barnes et al., J. Biol. Chem., 272(17):11510-7 (1997).
Benvenisty and Neshif, Proc. Nat'l Acad. Sci. USA, 83:9551-9555 (1986).
Bhavsar et al., Genomics, 35(1):11-23 (1996).
Bosher et al., Nat. Cell. Biol., 2(2):E31-E36 (2000).
Botinelli et al., Circ. Res. 82:106-115 (1997).
Brand et al., J. Biochem. Cell. Biol., 29:1467-1470 (1997).
Braunwald, Heart Disease—a Textbook of Cardiovascular Medicine (1997).
Bristow, Cardiology, 92:3-6 (1999).
Cairns et al., Chest, 108(4):3805 (1995).
Campbell, In: Monoclonal Antibody Technology, Laboratory Techniques in Biochemistry and Molecular Biology, Vol. 13, Burden and Von Knippenberg, Eds. pp. 75-83, Amsterdam, Elseview (1984).
Caplen et al., Gene, 252:95-105 (2000).
Chang et al., Hepatology, 14:124A (1991).
Chen and Okayama, Mol. Cell. Biol., 7:2745-2752 (1987).
Chin et al., Genes Dev., 12:2499-2509 (1998).
Coffin, Retroviridae and Their Replication. In: Virology, Fields et al., eds., Raven Press, New York, pp. 1437-1500 (1990).
Conte et al., J Heart Lung Transplant., 17:679-685 (1998).
Cook et al., Cell, 27:487-496 (1981).
Couch et al., Am. Rev. Resp. Dis., 88:394-403 (1963).
Coupar et al., Gene, 68:1-10 (1988).
Crabtree, G. R. and Olson, E., Cell, 109:S67-S79 (2002).
Davies et al., Clin. Biochem., 30:479-490 (1997).
De Feo et al., Ital. Heart J., 4:511-513 (2003).
DiBianco, R., Am. J. Med., 115:480-488 (2003).
Dolmetsch et al., Nature, 386:855-858 (1997).
Dresdale et al., Am. J. Med., 11(6): 686-705 (1951).
Dubensky et al., Proc. Nat'l Acad. Sci. USA, 81:7529-7533 (1984).
Dumcius et al., Medicina, 39:815-822 (2003).
Dunn et al., J. Biol. Chem., 274:21908-21912 (1999).
Durand et al., Ann. Med., 27:311-317 (1995).
Eichhorn and Bristow, Circulation, 94:2285-2296 (1996).
Elbashir et al., EMBO, 20(23):6877-6888 (2001).
Emmel et al., Science, 246:1617-1620 (1989).
Epstein, In The Metabolic and Molecular Bases of Inherited Disease: in Down Syndrome (Trisomy 21), Scriver, Beaudet, Valle, (eds), 7th Ed., Vol., pp. 749-794, McGraw-Hill, Inc., New York, 1995.
Eur. Pat. App. No. 0273085
Eur. Pat. No. 1,123,111.
Eur. Pat. No, 1,170,008.
Eur. Pat. No. 1,173,562.
Eur. Pat. No. 1,174,438.
Eur. Pat. No. 1,208,086.
Eur. Pat. No. 1,233,958.
Fechheimer, et al., Proc Nat'l. Acad. Sci. USA 84:8463-8467 (1987).
Ferkol et al., FASEB J., 7:1081-1091 (1993).
Fire et al., Nature, 391:806-811 (1998).
Forster and Symons, Cell, 49:211-220 (1987).
Fraley, et al., Proc Nat'l. Acad. Sci. USA 76:3348-3352 (1979).
Franz et al., Cardoscience, 5(4):235-43 (1994).
Friedmann, Science, 244:1275-1281 (1989).
Fuentes, et al., Genomics, 44:358-361 (1997).
Fuentes, et al., Hum. Mol. Genet., 4:1935-1944 (1995).
Furumai et al., Cancer Res., 62:4916-21 (2002).
Gao et al., J. Biol. Chem., 277:25748-55 (2002).
Genesca et al., Biochem. J., 374:567-575 (2003).
Gefter, et al., Somatic Cell Genet., 3:231-236 (1977).
Gerlach et al., Nature (London), 328:802-805 (1987).
Ghosh and Bachhawat, In: Liver Diseases, Targeted Diagnosis and Therapy Using Specific Receptors and Ligands. Wu et al., eds., Marcel Dekker, New York, pp. 87-104 (1991).
Ghosh-Choudhury et al., EMBO J., 6:1733-1739 (1987).
Goding, 1986, In: Monoclonal Antibodies: Principles and Practice, 2d ed., Academic Press, Orlando, Fla., pp. 60-61, and 71-74 (1986).
Gomez-Foix et al, J. Biol. Chem., 267:25129-25134 (1992).
Goodman and Gilman's “The Pharmacological Basis of Therapeutics.”
Gopal, Mol. Cell. Biol., 5:1188-1190 (1985).
Gopal-Srivastava et al., J. Mol. Cell. Biol., 15(12):7081-90 (1995).
Gottlicher et al., EMBO J., 20:6969-78 (2001).
Graham et al., J. Gen. Virol., 36:59-72 (1977).
Graham and Prevec, In: Methods in Molecular Biology: Gene Transfer and Expression Protocol, E. J. Murray, ed., Humana Press, Clifton, N.J., 7:109-128 (1991).
Graham and van der Eb, Virology, 52:456-467 (1973).
Grishok et al., Science, 287:2494-2497 (2000).
Grozinger et al., Proc. Natl. Acad. Sci. USA, 96:4868-4873 (1999).
Grunhaus and Horwitz, Seminar in Virology, 3:237-252 (1992).
Gruver et al., Endocrinology, 133:376-388 (1993).
Han et al., Cancer Research, 60:6068-6074 (2000).
Harland and Weintraub, J. Cell Biol., 101:1094-1099 (1985).
Harlow and Lane, Antibodies: A Laboratory manual, Cold Spring Harbor Laboratory (1988).
Hermonat and Muzycska, Proc. Nat'l Acad. Sci. USA, 81:6466-6470 (1984).
Hersdorffer et al., DNA Cell Biol., 9:713-723 (1990).
Herz and Gerard, Proc. Nat'l. Acad. Sci. USA 90:2812-2816 (1993).
Hill et al., J. Biol. Chem., 277:10251-10255 (2002).
Hinnebusch et al., J. Nutr., 132:1012-7 (2002).
Ho et al., J. Biol. Chem., 270:19898-19907 (1995).
Hoffmann et al., Bioconjugate Chem., 12:51-55 (2001).
Hoey et al., Immunity, 2:461-472 (1995).
Horwich, et al., J. Virol., 64:642-650 (1990).
Japanese Patent Application No. 2001/348340.
Jones and Shenk, Cell, 13:181-188 (1978).
Joyce, Nature, 338:217-244 (1989).
Jung, Curr. Med. Chem., 8:1505-11 (2001).
Jung et al., J. Med. Chem., 42:4669-4679 (1999).
Jung et al., Med. Chem. Lett., 7:1655-1658 (1997).
Kaneda et al., Science, 243:375-378 (1989).
Kao et al., Genes Dev., 14:55-66 (2000).
Karlsson et al., EMBO J., 5:2377-2385 (1986).
Kashishian et al., J. Biol. Chem., 273:27412-27419 (1998).
Kato et al., J Biol Chem., 266(6):3361-3364 (1991).
Kelly et al., J. Cell Biol., 129(2):383-96 (1995).
Ketting et al., Cell, 99(2):133-141 (1999).
Kim et al., Oncogene, 18:2461-2470 (1999).
Kim and Cook, Proc. Nat'l Acad. Sci. USA, 84:8788-8792 (1987).
Kimura et al., Dev. Growth Differ., 39(3):257-65 (1997).
Kitazomo et al., J. Clinical Endoc. MetaboL., 86(7):3430-3435 (2001).
Klee et al., J. Biol. Chem., 273:13367-13370 (1998).
Klein et al., Nature, 327:70-73 (1987).
Kohler and Milstein, Nature, 256:495-497 (1975).
Kohler and Milstein, Eur. J. Immunol., 6:511-519 (1976).
Komastsu et al., Cancer Res., 61:4459-4466 (2001).
Kramer et al., Trends in Endoc. Metabolism, 12)₇):294-300 (2001).
Kudoh et al., Circ. Res., 80:139-146 (1997).
Lai et al., J. Biol. Chem., 273:18325-18331 (1998).
LaPoint et al., Hypertension, 27:715-722 (1995).
LaPointe, et al., J. Biol. Chem., 263(19):9075-8 (1988).
Le Gal La Salle et al., Science, 259:988-990 (1993).
Levrero et al., Gene, 101:195-202 (1991).
Lin et al., Genetics, 153:1245-1256 (1999).
Lin et al., J. Clin. Invest., 97:2842-2848 (1996).
Liu et al., EMBO J., 16:143-153 (1997).
Macejak and Sarnow, Nature, 353:90-94 (1991).
Mai et al., J. Med. Chem., 45:1778-1784 (2002).
Mann et al., Cell, 33:153-159 (1983).
Manoria and Manoria, J. Indian. Med. Assoc., 101(5):311-312 (2003).
Mao et al., Science, 286:785-790 (1999).
Mao and Wiedmann, J. Biol. Chem., 274:31102-31107 (1999).
Markowitz et al., J. Virol, 62:1120-1124 (1988).
Massa et al., J. Med. Chem., 44:2069-2072 (2001).
Masuda et al., Mol. Cell. Biol., 15:2697-2706 (1995).
McCaffery et al., Science, 262:750-754 (1993).
Merck Index, Thirteenth Edition.
Mesaeli et al., J. Cell Biol., 144:857-868 (1999).
Michel and Westhof, J. Mol. Biol., 216:585-610 (1990).
Miyazaki, et al., J Biol. Chem., 271:14567-14571 (1996).
Molkentin et al., Cell, 93:215-228 (1998).
Montgomery et al., Proc. Nat'l. Acad. Sci., 95:15502-15507 (1998).
Moss et al., J. Gen. Physiol., 108(6):473-84 (1996).
Musaro et al., Nature, 400:581-585 (1999).
Nicolas and Rubinstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176 (1987).
Nicolau and Sene, Biochim. Biophys. Acta, 721:185-190 (1982).
Northrop et al., Nature, 369:497-502 (1994).
Olson, E. and Williams, R. S., Cell, 101:689-692 (2000).
Olson, E. and Williams, R. S., Bioassays, 22:510-519 (2000).
Olson et al., Dev. Biol., 172(1):2-14 (1995).
Oudiz et al., at www.emedicine.com/med/topic1962.htm, visited May 25, 2004.
Palmiter and Solaro, Basic. Res. Cardiol., 92:63-74 (1997).
Park et al., J. Biol. Chem., 271:20914-20921 (1996).
Paskind et al., Virology, 67:242-248 (1975).
PCT Application No. WO 84/03564.
PCT Application No. WO 98/33791.
PCT Application No. WO 99/32619.
PCT Application No. WO 00/44914.
PCT Application No. WO 01/14581.
PCT Application No. WO 01/18045.
PCT Application No. WO 01/36646.
PCT Application No. WO 01/38322.
PCT Application No. WO 01/42437.
PCT Application No. WO 01/68836.
PCT Application No. WO 01/70675.
PCT Application No. WO 02/26696.
PCT Application No. WO 02/26703.
PCT Application No. WO 02/30879.
PCT Application No. WO 02/46129.
PCT Application No. WO 02/46144.
PCT Application No. WO 02/50285.
PCT Application No. WO 02/51842.
Pelletier and Sonenberg, Nature, 334:320-325 (1988).
Perales, et al., Proc. Nat'l Acad. Sci. USA, 91(9):4086-4090 (1994). Physicians Desk Reference.
Potter et al., Proc. Nat'l Acad. Sci. USA, 81:7161-7165 (1984).
Racher et al., Biotechnology Techniques, 9:169-174 (1995).
Ragot et al.,” Nature, 361:647-650 (1993).
Rao et al., Annu Rev Immunol., 15:707-747 (1997).
Reinhold-Hurek and Shub, Nature, 357:173-176 (1992).
Remington's Pharmaceutical Sciences.
Renan, Radiother. Oncol., 19:197-218 (1990).
Rich et al., Hum. Gene Ther., 4:461-476 (1993).
Ridgeway, Mammalian Expression Vectors, In: Vectors: A Survey of Molecular Cloning Vectors and Their Uses, Rodriguez et al., eds., Stoneham: Butterworth, pp. 467-492, 1988.
Rippe, et al., Mol. Cell. Biol., 10:689-695 (1990).
Rooney et al., EMBO J., 13:625-633 (1994).
Rosenfeld, et al., Cell, 68:143-155 (1992).
Rosenfeld et al., Science, 252:431-434 (1991).
Rothermel et al., Trends Cardiovasc. Med., 13:15-21 (2003).
Rothermel et al., Proc. Natl. Acad. Sci. USA, 98:3328-3333 (2001).
Roux et al., Proc. Nat'l Acad. Sci. USA, 86:9079-9083 (1989).
Rosenfeld, et al., Science, 252:431-434 (1991).
Sadoshima et al., Cell, 75:977-984 (1993)
Sadoshima and Izumo, Circ. Res., 73:424-438 (1993).
Sarver et al., Science, 247:1222-1225 (1990).
Saunders et al., Cancer Res., 59-399-409 (1999).
Scanlon et al., Proc. Nat'l Acad. Sci. USA, 88:10591-10595 (1991).
Sharp et al., Science, 287:2431-2433 (2000).
Sharp, P. A., Genes. Dev., 13:139-141 (1999).
Semsarian et al., Nature, 400:576-581 (1999).
Sigal et al., J. Exp. Med., 173:619-628 (1991).
Stemmer and Klee, Biochemistry, 33:6859-6866 (1994).
Stratford-Perricaudet et al., Hum. Gene. Ther., 1:241-256 (1990).
Stratford-Perricaudet and Perricaudet, In: Human Gene Transfer, O. Cohen-Haguenauer et al., eds., John Libbey Eurotext, France, pp. 51-61 (1991).
Su et al., Cancer Res., 60:3137-3142 (2000).
Su et al., Eur. J. Biochem., 230:469-474 (1995).
Sun et al., Immunity, 8:703-711 (1998).
Sussman et al., Science, 281:1690-1693 (1998).
Tabara et al., Cell, 99(2):123-132 (1999).
Takahashi et al., Antibiotics, 49:453 (1996).
Taunton et al., Science, 272:371 (1996).
Temin, In: Gene Transfer, Kucherlapati R, ed., New York, Plenum Press, pp. 149-188, 1986.

Timmerman et al., Nature, 383(6603):837-40, (1996).
Tong et al., Nucleic Acids Res., 30:1114-23 (2002).
Top et al., J. Infect. Dis., 124:155-160 (1971).
Tur-Kaspa, et al., Mol. Cell. Biol., 6:716-718 (1986).
United States App. 2002/61860
United States App. 2002/65282
United States App. 2002/103192
United States App. 2002/150953
United States App. 2002/256221
U.S. Pat. No. 5,888,773
U.S. Pat. No. 5,889,136
U.S. Pat. No. 5,795,715
U.S. Pat. No. 5,708,158
U.S. Pat. No. 5,604,251
U.S. Pat. No. 5,354,855
U.S. Pat. No. 4,946,778
U.S. Pat. No. 4,458,066
U.S. Pat. No. 4,415,732
U.S. Pat. No. 4,265,874
U.S. Pat. No. 4,256,108
U.S. Pat. No. 4,196,265
U.S. Pat. No. 4,166,452
Van den Wyngaert et al., FEBS Lett., 478:77-83 (2000).
Varmus et al., Cell, 25:23-36 (1981).
Vigushin et al., Anticancer Drugs, 13: 1-13 (2002).
Vigushin et al., Clinical Cancer Res., 7:971-976 (2001).
Vigushin et al., Cancer Res., 5S (1999).
Vikstrom and Leinwand, Curr. Opin. Cell Biol., 8:97-105 (1996).
Wagner, et al., Proc. Nat'l Acad. Sci. USA 87(9):3410-3414 (1990).
Watkins et al., Hum. Mol. Genet., 4:1721-1727 (1995).
Wincott et al., Nucleic Acids Res., 23:2677-2684 (1995).
Wolfe et al., Nature, 385:172-176 (1997).
Wong et al., Gene, 10:87-94 (1980).
Workman and Kingston, Annu. Rev. Biochem., 67:545-579 (1998).
Wu and Wu, Adv. Drug Delivery Rev., 12:159-167 (1993).
Wu and Wu, Biochemistry, 27:887-892 (1988).
Wu and Wu, J. Biol. Chem., 262:4429-4432 (1987).
Yamauchi-Takihara, et al., Proc. Nat'l Acad. Sci. USA, 86(10):3504-8 (1989).
Yamazaki et al., Circulation, 95:1260-1268 (1997).
Yamano et al., Amer. Soc. Gene Ther. (2000).
Yang, et al., Proc Nat'l Acad. Sci. USA, 87:9568-9572 (1990).
Young et al., Handbook of Applied Therapeutics, 7.1-7.12 and 9.1-9.10 (1989).
Zelenin et al., FEBS Lett., 280:94-96 (1991).
Zhou et al., Proc. Natl. Acad. Sci., 98:10572-10577 (2001).
Ziober and Kramer, J. Bio. Chem., 271(37):22915-22 (1996).
Zou et al., J. Biol. Chem., 271:33592-33597 (1996).

1. A method of treating cardiovascular disease comprising:
(a) identifying a patient having cardiovascular disease; and
(b) administering to said patient a modulator of MCIP-1-38.

2. The method of claim 1, wherein said cardiovascular disease comprises one or more of pathologic cardiac hypertrophy, dilated cardiomyopathy, myocardial infarction, primary or secondary pulmonary arterial hypertension, chronic heart failure, ischemic heart disease.

3. The method of claim 1, wherein said modulator is a small molecule, a peptide, a protein, a cyclic peptide, or a pharmaceutical.

4. The method of claim 1, wherein said modulator is a nucleic acid.

5. The method of claim 4, wherein said nucleic acid is an siRNA, an antisense RNA, or encoded by a viral expression vector.

6. The method of claim 1, wherein administering comprises intravenous administration of said modulator.

7. The method of claim 1, wherein administering comprises oral, transdermal, sustained release, suppository, sublingual, subcutaneous, direct injection, stent, or a gene therapy administration of said modulator.

8. The method of claim 1, further comprising administering to said patient a second therapeutic regimen.

9. The method of claim 8, wherein said second therapeutic regimen is selected from the group consisting of a beta blocker, an inotrope, phosphodiesterase inhibitors, diuretic, ACE-inhibitor, All antagonist, histone deacetylase inhibitor, a Ca(++)-channel blocker, endothelin receptor antagonists.

10. The method of claim 8, wherein said second therapeutic regimen is administered at the same time as said modulator.

11. The method of claim 8, wherein said second therapeutic regimen is administered either before or after said modulator.

12. The method of claim 1, wherein treating comprises improving one or more symptoms of cardiac hypertrophy.

13. The method of claim 12, wherein said one or more symptoms comprises a dysfunction in any one of exercise capacity, blood ejection volume, left ventricular end diastolic pressure, pulmonary capillary wedge pressure, cardiac output, decreased cardiac index, pulmonary artery pressures, left ventricular end systolic and diastolic dimensions, left and right ventricular wall stress, or wall tension, quality of life, disease-related morbidity and mortality, or decreased hospitalizations.

14. The method of claim 1, wherein treating comprises improving one or more symptoms of heart failure.

15. The method of claim 14, wherein one or more symptoms comprises heart failure related hospitalizations, decreased exercise capacity, progressive remodeling, ventricular dilation, decreased cardiac output, impaired pump performance, arrhythmia, fibrosis, necrosis, energy starvation, and apoptosis.

16. A method of preventing cardiac hypertrophy or heart failure comprising:
(a) identifying a patient at risk for cardiac hypertrophy or heart failure; and
(b) administering to said patient a modulator of MCIP-1-38.

17. The method of claim 16, wherein administering comprises intravenous administration of said modulator.

18. The method of claim 17, wherein administering comprises oral, transdermal, sustained release, suppository, sublingual, subcutaneous, direct injection, stent, or a gene therapy administration of said modulator.

19. The method of claim 16, wherein the patient at risk may exhibit one or more of long standing uncontrolled hypertension, atherosclerosis, uncorrected valvular disease, chronic angina and/or recent myocardial infarction.

20. The method of claim 16, wherein said modulator consists of a small molecule, a peptide, a protein, a cyclic peptide, or a pharmaceutical.

21. The method of claim 16, wherein said modulator is a nucleic acid.

22. A method of identifying a modulator of MCIP-1-38 comprising:
(a) providing a cell;
(b) contacting said cell with a candidate substance; and
(c) measuring expression of MCIP-1-38;
wherein an increase in expression of MCIP-1-38, as compared to expression in an untreated cell, identifies the candidate substance as a modulator of MCIP-1-38.

23. The method of claim 22, wherein said cells are cardiomyocytes.

24. The method of claim 22, wherein said MCIP-1-38 is measured from intact cells, and wherein the expressed MCIP-1-38 gene is either native or exogenous.

25. The method of claim 22, wherein said cardiomyocytes are selected from neonatal rat ventricular myocytes, adult rat cardiomyocytes, neonatal mouse cardiomyocytes, adult mouse cardiomyocytes, or adult human cardiomyocytes.

26. The method of claim 22, wherein said cardiomyocytes are located in an intact mammalian heart.

27. The method of claim 26, wherein said heart is a rat heart, a mouse heart, or a human heart.

28. The method of claim 22 further comprising a high-throughput screening method.

29. A method of identifying an inhibitor of heart failure or hypertrophy comprising:
(a) providing an MCIP-1-38 modulator;
(b) treating a myocyte with said modulator; and
(c) measuring the expression of one or more cardiac hypertrophy or heart failure parameters,
wherein a change in said one or more cardiac hypertrophy or heart failure parameters, as compared to one or more cardiac hypertrophy parameters in a myocyte not treated with said enhancer, identifies said modulator as an inhibitor of heart failure or cardiac hypertrophy.

30. The method of claim 29, wherein said myocyte is subjected to a stimulus that triggers a hypertrophic response in said one or more cardiac hypertrophy parameters.

31. The method of claim 30, wherein said stimulus is expression of a transgene.

32. The method of claim 30, wherein said stimulus is treatment with a chemical agent.

33. The method of claim 29, wherein said one more cardiac hypertrophy parameters comprises the expression level of one or more target genes in said myocyte, wherein expression level of said one or more target genes is indicative of cardiac hypertrophy.

34. The method of claim 33, wherein said one or more target genes is selected from the group consisting of ANF, α-MyHC, β-MyHC, α-skeletal actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen.

35. The method of claim 29, wherein the expression level is measured using a reporter protein coding region operably linked to a target gene promoter.

36. The method of claim 35, wherein said reporter protein is luciferase, β-gal, or green fluorescent protein.

37. The method of claim 29, wherein the expression level is measured using hybridization of a nucleic acid probe to a target mRNA or amplified nucleic acid product.

38. The method of claim 29, wherein said one or more cardiac hypertrophy parameters comprises one or more aspects of cellular morphology.

39. The method of claim 38, wherein said one or more aspects of cellular morphology comprises sarcomere assembly, cell size, cellular fusion, or cell contractility.

40. The method of claim 29, wherein said myocyte is an isolated myocyte.

41. The method of claim 29, wherein said myocyte is contained in isolated intact tissue.

42. The method of claim 29, wherein said myocyte is a cardiomyocyte.

43. The method of claim 42, wherein said cardiomyocyte is a neonatal rat ventricular myocyte.

44. The method of claim 42, wherein said cardiomyocyte is located in vivo in a functioning intact heart muscle.

45. The method of claim 44, wherein said functioning intact heart muscle is subjected to a stimulus that triggers heart failure or a hypertrophic response in one or more cardiac hypertrophy parameters.

46. The method of claim 45, wherein said stimulus is induced by aortic banding, rapid cardiac pacing, induced myocardial infarction, a drug-containing osmotic minipump, or by transgene expression.

47. The method of claim 46, wherein said one or more cardiac hypertrophy parameters comprises right ventricle ejection fraction, left ventricle ejection fraction, ventricular wall thickness, heart weight/body weight ratio, heart weight/tibia length, heart weight/brain weight ratio, heart weight to bone length ratios, or cardiac weight normalization measurement.

48. The method of claim 29, wherein said one or more cardiac hypertrophy parameters comprises total protein synthesis.

49. A polyclonal antisera that binds immunologically to MCIP-1-38.

50. A method for monitoring MCIP-1-38 protein levels in periperhal blood comprising:
(a) isolating PBMCs from whole blood of an individual
(b) analyzing the cells for the presence of MCIP-1-38 using an antibody or antisera that binds immunologically to MCIP-1-38.

51. The method of claim 50, wherein analyzing comprises lysing the cells and running an ELISA on the lysate.

52. The method of claim 50, wherein analyzing comprises western blot analysis of cell lysate.

53. The method of claim 50, wherein analyzing comprises performing an in situ ELISA on intact cells.

54. A kit for detecting MCIP-1-38 protein levels in peripheral blood comprising an MCIP-1-38 antibody and reagents and controls necessary for analyzing MCIP-1-38 levels.

55. The kit of claim 54, wherein reagents and controls comprise reagents and controls needed to run an ELISA, a western blot, or an in situ ELISA on intact cells.
